SEQUENCE LISTING (12 sequences)

SEQ ID NO: 1 - DNA, 22 bp, artificial
  Primer
agcagttcccttcagcaaaaat

SEQ ID NO: 2 - DNA, 22 bp, artificial
  primer
gaatcggcggtatcatccttat

SEQ ID NO: 3 - DNA, 24 bp, artificial
  mutagenic nucleobase
atcatcctacgttgcacttgaccg

SEQ ID NO: 4 - DNA, 24 bp, artificial
  mutagenic nucleobase
atcatcctcctctgcacttgaccg

SEQ ID NO: 5 - DNA, 24 bp, artificial
  mutagenic nucleobase
aucauccucctctgcacuugaccg

SEQ ID NO: 6 - DNA, 24 bp, artificial
  mutagenic nucleobase
guacguacagucaguacguaggau

SEQ ID NO: 7 - DNA, 23 bp, Artificial
  primer
cctggtctattggatattgttag

SEQ ID NO: 8 - DNA, 20 bp, artificial
  Primer
gcttgagcagttctgtattc

SEQ ID NO: 9 - DNA, 22 bp, artificial
  primer
gcaatcaaggaggaatcagagg

SEQ ID NO: 10 - DNA, 20 bp, artificial
  primer
ccagcagcatcaatcaagcc

SEQ ID NO: 11 - DNA, 2505 bp, Lycopersicon esculentum
atggaagacgaagccattccagtgccgattccgaaggagccaccgaagattcagcggctggaagaatgtgtggtgaacagaatagcggctggcgaagtcatccaaaggccagtctctgccgtgaaagagctcattgagaacagcctggatgctgattccacctctatttccgttgttgttaaggatggcggtcttaaacttatccaagtttccgacgatggccatggaatccgttatgaagatttgccaattttatgcgagaggtatactacgtccaagctgagtaaatttgaagatttgcagtccattaggtcgatgggatttagaggagaagccttggctagcatgacatatgtgggtcacgtcactgtcaccaccattactatgggccagttgcatggatacagggcaacatatagagatggtttgatggtggatgagccaaaggcttgtgctgctgtcaagggtacccagataatgattgaaaatttattttataacatggctgcacgaaggaaaacccttcaaaattctgccgatgactatccaaaaattgttgacattattagtaggtttggaattcatcacacacatgtgagcttctcttgtagaaagcatggagctggtagagcagatgttcacactattgctacttcttcaaggctcgatgcaattagatccgtttatggagcttcagttgctcgagatctgatgaatatcgaagtttctgatactggtccattaatttcagtttttaagatggatggtttcatctccaactctaattatattgcgaagaagacaacaatggtgctttttataaatgatagactcattgattgtggtgctttgaagagggcaattgaaatagtctatactgcaacattgcctaaagcatcaaaacctttcatatacatgtcaatcattttgccgcccgagcatgttgatgtgaatatacacccaacaaagagagaggtaagctttttgaatcaagagttcgtcattgagaagatccagtctgtagtagggtcaaaattgagaagctccaatgagtcgaggacattccaggaacagactatggatttatcttcatctggtccaatgggccaagattccactaaagaatcgtctccttctgggataaagtcacaaaaagtgccacataaaatggtacgaacagatactttggacccttctggaaggctgcacgcttacatgcaaatgaagcctcctggtaattcagaaagaggtccttgctttagctctgtgaggtcttctatcagacaaaggaggaatcctagtgacaccgcagacctcactagcatccaagagctcgttaatgagattgataatgactgtcaccctggtctattggatattgttaggaattgcacatatactgggatggcggatgagatttttgctttgcttcaacacaatacacacctttatcttgttaatgtgattaacttgagtaaagagcttatgtatcagcaagttttacgtcggtttgcccatttcaatgcaattcaactgagtgaaccagcatcattacctgagttagtaatgcttgctctgaaagaagagggttcagatccagaaggcaacgaaagcaaagagctaagaggaaagattgccgagattgaatacagaactgctcaagcaaaaggctggaatgctagaaggagtattttagtattcattatcgattcaaatggaaatatgtctagtcttcctgtatactgggatcagtacacacctgacatgggaccgcatcccagaaatttattactttggttcaggaaaattttgaactggggaaggacgaaaaaatttggttttcaagacaattggctggtggtcctaaggaaaatttttttgccatgcattccgccattattgcctaaatccctcaggggatggcttgaaattctacagaagagagaactttcaagtggttcagaagtaacttcaatagataacatagagaatgataccacggaggctgaatttgacgaagaactacgtttggaggctgaaaatgcctgggctcaacgtgaatggtcaattcagcatgttctgtttccctccctcaggctcttcttcaagccccctacttccatggttacaaatggaacttttgttcaggttgcatcactggaaaagctctacagaattttcgagagatgttaaacctaggagaattttgatgtggcctccttgaagcaaaaggatgtaaaatgtacagtttctgatttgagaaggaaattccctggcccctacccccaaagaaccaaaatatgaaaaactggttcaactttttttttggtgtgaagacaaccacccccctacatcaaacgttttattgtgaaaagaagcaaaaatggaaggaatacattttgtctcttttactctattccatcaaactagtggtgtagacatggtgttatacgcaagaaatgagttaagcaaataggagttcaaaagaaaaaaaaaaaaaaaaaa

SEQ ID NO: 12 - DNA, 2832 bp, Lycopersicon esculentum
atggatgaaaattttgaggaacagggcaagcttccagagcttaaacttgatgcgaggcaagctcaagggtttctttcattcttcaaaaccctacccaaggatgttagggcagttcgtctattcgatcgtagggactattatactgctcatggagatgatgcaactttcattgcaaagacatattaccatacgacaactgctttacggcagttgggtaatggagttggtgcgctttccagtgttagtgtgagtagaaacatgtttgaaacaatagctcgtgacattctcttggagaggatggatcgtactcttgaattgtatgagggcagtggttcaaattggaaactggtcaaaagtggaaccccaggaaatttcggaagttttgaggacattctgtttgctaataatgaaatgcaagattctccagtgattgttgctcttgcgccaaaatttgatcagaatggatgtacagttgggttaggctatgttgatattactaagagagtccttggtttagcagaatttctagatgatagccacttcaccaatttggagtctgctttggttgcccttggttgcagagaatgtcttgtaccaacagagactgggaaatccagtgagagcaggcctctatatgatgcaatatcgagatgcggggtgatggtaactgaaagaaagaaaactgaatttaaaggtagggatttggtacaggatctgggtaggcttgtcaagggttcagtagaacctgttcgagatctagtctctagttttgaatgtgcagcaggtgctttggggtgcatactttcctatgcagaattacttgcggatgacagcaattatggaaactacacagtcaaacaatacaacctcgatagttacatgagattagattctgctgctatgagagcactgaatgttatggagagcaaatcagatgctaataaaaattttagcttgtttggtctcatgaatagaacctgtactgctggaatgggtaaaaggttattgcacatgtggctgaaacagccgttactagatgtagatgagattaactgtagattggatttagttcaagcatttgtggaggatgctgcacttcgccaagatttgaggcagcatctgaaaagaatttcagatattgagcggctgacacacaatcttgagaggaaaagagccagtttattgcacgttgtaaaactctatcagtcaggcatcagaataccatatatcaaaagtgttttggaacgttatgatgggcaatttgcaccgctaatcagggaaaggtatattgattctcttgagaaatggagtgatgataatcatctgaataagttcattgctcttgtggaaactgctgttgaccttgatcaacttgagaatggagaatacatgatttcttctgcatatgacccaaatttatctgctctgaaggatgagcaagagacattggagcaacagattcataatttgcacaaacaaactgccaatgatcttgatctacctattgataagtctcttaaattagataaaggaacacaatttggacatgtctttagaattaccaagaaagaagaaccaaaagtcaggaggcagctaaactctcactacattgttcttgaaacacgcaaggatggggtaaaattcaccaatacaaaactcaaaaaactaggagatcggtaccagaagatcttagacgagtataagagctgtcagaaagaactggtagctcgggtagttcaaacagttgcgagtttctctgaggtatttgaaggtttagctggttcactttctgagttggatgtgctactgagttttgcggatttggcttccagttgcccaactgcctactcaagaccaaatatcagtccaccagatacgggagatattatacttgaagggtgtagacatccttgtgtggaagctcaagactgggtcaacttcatccctaatgactgtagactagttagaggagaaagttggtttcagattatcacaggccctaacatgggtggaaagtctacctacattcgtcaggttggtgtgaatgtcctgatggcccaagttggctcatttgttccatgtgacaatgctaccatttctattcgtgattgcatttttgctcgtgttggcgctggagattgtcagctgaagggggtttctacttttatgcaagagatgcttgagactgcatcgatcttgaaaggagctactaatagatcgttggttataattgatgagttgggccgtgggacgtcgacttatgatggctttggtttagcttgggctatttgtgagcacattgttgaagaaattaaagcaccaacattgtttgcaactcactttcatgagctgactgcattggccaatgagaatggaaacaatggacataagcaaatttccagtgtggcaaattttcatgtcagtgcacacattgactcttctagtcgcaagctaactatgctttacaaggttcaaccaggtgcttgtgatcaaagttttggtattcacgtagcagagtttgccaattttccacaaagtgttgtggccctggccagagaaaaggcttctgagttggaggatttctctcctcgtgctatgatgccaaatgactgttttcaggtagtctcaaagcggaagagggaatttgacccacatgatgtgtctagaggtactgcccgagctcgtcaattcttacaggatttcactcagttgccactggataagatggatctaaagcaggcgttgcaacagttgagccaaatgaagactgaccttgagaagaatgcagttgacagtcagtggcttcagcagttctttagttcttcaaattag

(19) United States
(12) Patent Application Publication
(10) Pub. No.: US 2011/0312094 A1
(43) Pub. Date: Dec. 22, 2011

(54) USE OF DOUBLE STRANDED RNA TO INCREASE THE EFFICIENCY OF TARGETED GENE ALTERATION IN PLANT PROTOPLASTS

(75) Inventor: Paul Bundock, Abcoude (NL)

(73) Assignee: Keygene N.V.

(21) Appl. No.: 13/141,196

(22) PCT Filed: Dec. 22, 2009
(86) PCT No.: PCT/NL2009/000270

§ 371 Date: Aug. 4, 2011

Related U.S. Application Data
(60) Provisional application No. 61/139,769, filed on Dec. 22, 2008.

(51) Int. Cl. C12N 15/01

(52) U.S. Cl. 435/442; 435/441

(57) ABSTRACT

Method for targeted gene alteration in protoplasts of plant cells comprising the steps of transiently transfecting the protoplasts with a dsRNA that preferably targets plant MMR mRNA; and a mutagenic nucleobase. The transfection may be simultaneously or subsequently and the gene can be any gene functional in the mismatch repair system.

### FIELD OF THE INVENTION

The present invention relates to biotechnology, in particular plant biotechnology. The invention relates more in particular to methods for targeted gene alteration of plant genes in protoplasts using mutagenic nucleobases in the presence of dsRNA molecules. The invention further relates to increasing the efficiency of targeted gene alteration and to the application of gene alteration using this technology.

### BACKGROUND OF THE INVENTION

Genetic modification is the process of deliberately creating changes in the genetic material of living cells with the purpose of modifying one or more genetically encoded biological properties of that cell, or of the organism of which the cell forms part or into which it can regenerate. These changes can take the form of deletion of parts of the genetic material, addition of exogenous genetic material, or changes in the existing nucleotide sequence of the genetic material. Methods for the genetic modification of eukaryotic organisms have been known for over 20 years, and have found widespread application in plant, human and animal cells and micro-organisms for improvements in the fields of agriculture, human health, food quality and environmental protection. The common methods of genetic modification consist of adding exogenous DNA fragments to the genome of a cell, which will then confer a new property to that cell or its organism over and above the properties encoded by already existing genes (including applications in which the expression of existing genes will thereby be suppressed). Although many such examples are effective in obtaining the desired properties, these methods are nevertheless not very precise, because there is no control over the genomic positions in which the exogenous DNA fragments are inserted (and hence over the ultimate levels of expression), and because the desired effect will have to manifest itself over the natural properties encoded by the original and well-balanced genome. On the contrary, methods of genetic modification that will result in the addition, deletion or conversion of nucleotides in predefined genomic loci will allow the precise modification of existing genes.

Mutagenic nucleobase directed targeted gene alteration (TGA) is a method that is based on the delivery into the eukaryotic cell nucleus of synthetic mutagenic nucleobases (molecules consisting of short stretches of nucleotide-like moieties that resemble DNA in their Watson-Crick basepairing properties, but may be chemically different from DNA) (Alexeev and Yoon, Nature Biotechnol. 16: 1343, 1998; Rice, Nature Biotechnol. 19: 321, 2001; Kmiec, J. Clin. Invest. 112: 632, 2003). By deliberately designing a mismatch nucleotide in the homology sequence of the mutagenic nucleobase, the mismatch nucleotide may be copied into the genomic DNA sequence. This method allows the conversion of single or at most a few nucleotides in existing loci, but may be applied to create stop codons in existing genes, resulting in a disruption of their function, or to create codon changes, resulting in genes encoding proteins with altered amino acid composition (protein engineering).

TGA has been described in plant, animal and yeast cells. Two different classes of synthetic mutagenic nucleobase have been used in these studies, the chimeric DNA:RNA type (chimeras) or the single stranded type. The chimeras are self complementary molecules consisting of a 25 by DNA only region and a 25 bp complementary sequence made up of 5 bp of core region of DNA flanked on either side by 10 bp of 2′-O-methylated RNA that are thought to aid stability of the chimera in the cell. The 5 bp core region includes in its centre an engineered mismatch with the nucleotide to be altered in the genomic target DNA sequence. Both these regions are linked by 4 by thymidine hairpins. Upon introduction into the cell the chimera is thought form a double D-loop with its target sequence and a mismatch is formed between the chimera and the target nucleotide. This mismatch is then resolved by endogenous cellular DNA repair proteins by conversion of the genomic nucleotide. The first examples of TGA using chimeras came from animal cells (reviewed in lgoucheva et al. 2001 Gene Therapy 8, 391-399) and were then also later used to achieve TGA in plant cells (Beetham et al. 1999 Proc. Natl. Acad. Sci. USA 96: 8774-8778; Zhu et al. 1999 Proc. Natl. Acad. Sci. USA 96, 8768-8773; Zhu et al. 2000 Nature Biotech. 18, 555-558; Kochevenko et al. 2003 Plant Phys. 132: 174-184; Okuzaki et al. 2004 Plant Cell Rep. 22: 509-512). Unlike human cells, a plant cell in which a TGA event has occurred can be regenerated into an intact plant and the TGA mutation transferred to the next generation, making it an ideal tool for both research and commercial genetic engineering of important food crops. However, extensive research by many laboratories has shown that the TGA frequency using chimeras is quite low and variable, or not even detectable (Ruiter et al. 2003 Plant Mol. Biol. 53, 715-729, Van der Steege et al. (2001) Nature Biotech. 19: 305-306), and depended on such factors as the transcriptional status of the target, the position of the cell in the cell cycle, the sequence of the target and the quality of the chimeras, which are difficult to synthesize. Due to the relatively low frequency of TGA, TGA events can only be detected when alteration of a single nucleotide results in a dominant selectable phenotype. In plant cells specific point mutations were introduced into the open reading frame of the acetolactate synthase (ALS, in maize AHAS) gene which catalyzes the initial step common to the synthesis of the branched chain amino acids leucine, isoleucine and valine. In tobacco, single nucleotide alterations are sufficient to produce the codon conversions P194Q or W571L. The ALS protein produced after either of these codon conversions is insensitive to inhibition by the sulfonylurea class of herbicides, thus providing a method of selection for single nucleotide conversions at a chromosomal locus.

Due to the difficulties of working with chimeras, more reliable alternative oligonucleotide designs have been sought. Several laboratories have investigated the ability of single stranded (ss) mutagenic nucleobases to perform TGA. These have been found to give more reproducible results, be simpler to synthesize, and can also include modified nucleotides to improve the performance of the mutagenic nucleobase in the cell (Liu et al. 2002 Nuc. Acids Res. 30: 2742-2750; review, Parekh-Olmedo et al. 2005 Gene Therapy 12: 639-646; Dong et al. 2006 Plant Cell Rep. 25: 457-65; De Piédoue et al. 2007 Oligonucleotides 27: 258-263).

TGA has been described in a variety of patent applications of Kmiec, inter alia in WO0173002, WO03/027265, WO01/87914, WO99/58702, WO97/48714, WO02/10364. In WO 01/73002 it is contemplated that the low efficiency of gene alteration obtained using unmodified DNA oligonucleotides is largely believed to be the result of degradation of the donor oligonucleotides by nucleases present in the reaction mixture or the target cell. To remedy this problem, it is proposed to incorporate modified nucleotides that render the resulting mutagenic nucleobase resistant against nucleases. Typical examples include nucleotides with phosphorothioate linkages or 2′-O-methyl-analogs. These modifications are preferably located at the ends of the mutagenic nucleobase, leaving a central DNA domain surrounding the targeted base. In support of this, patent application WO 02/26967 shows that certain modified nucleotides increasing the intracellular lifetime of the mutagenic nucleobase enhance the efficiency of TGA in an in vitro test system and also at a mammalian chromosomal target. Not only the nuclease resistance, but also the binding affinity of a mutagenic nucleobase to its complementary target DNA has the potential to enhance the frequency of TGA dramatically. A single stranded mutagenic nucleobase containing modified nucleotides that enhance its binding affinity may more efficiently find its complementary target in a complex genome and/or remain bound to its target for longer and be less likely to be removed by proteins regulating DNA transcription and replication. An in vitro TGA assay has been used to test many modified nucleotides to improve the efficiency of the TGA process. Locked nucleic acids (LNA) and C5-propyne pyrimidines have modifications of the sugar moiety and base respectively that stabilize duplex formation and raise the melting temperature of the duplex. When these modified nucleotides are incorporated on a mutagenic nucleobase, they enhance the efficiency of TGA up to 13 fold above that obtained using an unmodified mutagenic nucleobase of the same sequence. See in his respect WO2007073166 and WO2007073170.

Studies in animal and yeast cells have shown that proteins belonging to the cellular mismatch repair (MMR) system are important in the TGA process. During DNA replication, occasionally the DNA dependent DNA polymerase incorporates the incorrect nucleotide on the newly synthesized (daughter) DNA strand. This results in a mismatch of nucleotides (e.g. G:A, T:C, G:G etc) in the DNA duplex which must be corrected to maintain the genetic integrity of the cell. The MMR complex in E. coli consists of 3 classes of subunits, the MutS, MutL and MutH proteins. There are several MutS proteins that function as heterodimers and are able to bind to mismatches in the DNA duplex. These MutS heterodimers differ in their affinity for different mismatches. Once bound to the mismatch, the MutS heterodimer recruits the MutL heterodimers to the mismatch, which in turn recruits the MutH protein. MutH is able to nick the newly synthesized DNA strand close to and on one side of the mismatch. Beginning at the nick, an exonuclease is then able to begin degradation of the newly synthesized DNA, including the mismatched nucleotide. The repair of the mismatch is then completed by re-synthesis of the daughter strand. The MMR system is ubiquitous and orthologs of MutS and MutL proteins have been found in both prokaryotic and eukaryotic genomes, including those of animals and plants (for review see Kolodner & Marsishky 1999, Curr. Opin. Genet. Dev. 9: 89-96). In plants, four MutS orthologs (MSH2, MSH3, MSH6 and MSH7) and four MutL orthologs (MLH1, MLH2, MLH3 and PMS1) are present. Mismatch recognition of base-base mispairs or single extrahelical nucleotides is accomplished by MutSα (a MSH2::MSH6 heterodimer) while larger extrahelical loopouts are recognized by MutSβ (MSH2::MSH3 heterodimer). The MSH7 gene has been identified in plants but not thus far in animals. MSH7 is most similar to MSH6 and also forms a heterodimer (MutSγ) with MSH2 (Culligan & Hays, 2000, Plant Cell 12: 991-1002). However, the MutSα and MutSγ exhibit somewhat different affinities for the range of mismatches. Cells lacking MSH2 are unable to recognize DNA mismatches, and show a mutator phenotype. In Arabidopsis lines lacking MSH2, mutations accumulate per generation up to a point (T6 generation) at which the plants lose viability (Hoffman et al. 2004 Genes & Dev. 18: 2676-2685). In the moss Physcomitrella, loss of MSH2 results immediately in deleterious phenotypes, probably due to the haploid nature of this plant (Trouiller et al. 2006 Nuc. Acids Res. 34: 232-242). Genetic lesions in Arabidopsis MSH2 mutants have also been detected in microsatellites, which are hyper mutable regions of the genome (Leonard et al. 2003 Plant Phys. 133: 328-338; Depeiges et al. 2005 Plant Sci. 168: 939-947). In addition, MSH2 mutants show increased somatic and meiotic homologous recombination between divergent sequences (Emmanuel et al. 2005 EMBO Rep. 7: 100-105; Li et al. 2006 Plant J. 45: 908-916), indicating that recombination between non-identical sequences is inhibited by the MMR system.

The MutL orthologs form the following heterodimers, MutLα (MLH1::PMS1), MutLβ (MLH1::MLH3) and MutLγ (MLH1::MLH2) and each heterodimer is involved in the repair of a different DNA lesion. MLH1 is obviously very important as it is involved in all the heterodimers but PMS1 also plays an important role as, part of the major MutLα heterodimer, it is involved in the repair of single mispaired bases. The Arabidopis PMS1 gene has been recently identified (Alou et al. 2004 Plant Sci. 167: 447-456). As with all the MMR genes, PMS1 expression is very low in mature plant tissues, but highly upregulated in dividing cell cultures as would be expected due to its role in the repair of DNA replication errors. Plants lacking PMS1 show the same microsatellite instability as plants lacking MSH2, indicating that loss of MutLα function is sufficient to give a mutator phenotype (Alou et al. 2004 Plant Mol. Biol. 56: 339-349).

It has been clearly demonstrated that the MMR system inhibits the TGA process in animal cells. Dekker et al. (2003 Nuc. Acids Res. 31: e27) performed TGA experiments in mouse embryonic stem (ES) cells and showed that the single nucleotide substitutions could only be obtained in ES lines lacking MSH2. Dekker et al. (2006 Gene Therapy 13: 686-694) also found similar results in ES lines lacking MSH3. In addition, Igoucheva et al. (2008 Oligonucleotides 18: 111-122) demonstrated in liver hepatocytes that restoration of a chromosomally integrated GFP reporter gene by a single nucleotide substitution was 30 fold more efficient in lines in which MSH2 expression was suppressed using RNAi. Transient suppression of the MMR system using RNAi has also been shown to improve the TGA efficiency. Maguire et al. (2007 Gene 386: 107-114) co-transformed a plasmid carrying a defective GFP gene, a mutagenic nucleobase designed to correct this mutation and a siRNA targeted to the MSH2 transcript. Even though the level of MSH2 downregulation was limited (only 62% of control expression) they observed a 3 fold improvement in the TGA frequency. There are also reports that mutations in the MutL heterodimers also lead to an increase in the efficiency of TGA. Yin et al. (2005 Biochem. J. 390: 253-261) reported that the frequency of TGA at the endogenous β-globin gene was increased by 5 fold in a human colon cancer line lacking MLH1 activity. Analogous with MSH2, MLH1 is present in all of the MutL heterodimers. The simplest explanation for the increase in TGA efficiency seen in MMR-deficient animal cell lines is that the mismatch formed between the mutagenic nucleobase and its target is detected by the functional MMR system and the TGA process is aborted. Given the wide range of cell types used in these studies, it appears that the inhibition of TGA by the MMR-system is not limited to specific cell types. Plant cells lacking a MMR system may hence also show an increase in the TGA efficiency. However, it is clear that it is not desirable to use plant lines with a permanent down regulation of the MMR system as they will continue to accumulate DNA-replication associated errors and will eventually become unviable. Thus, a method to transiently down regulate the MMR system in plant cells is desirable. However, such a system is not yet available in the art.

The use of dsRNA in the transient suppression of the MMR system in plant protoplasts has thus far not been described, suggested or attempted.. The use of dsRNA in the transient suppression of the MMR system in plant protoplasts to increase the efficiency of TGA has also not been dislcosed or suggested in the art.

### SUMMARY OF THE INVENTION

The present inventors have found that the efficiency of TGA with a mutagenic nucleobase in plant cells is significantly improved by the transient suppression of the MMR system in plant protoplasts. The invention thus involves transfection of, preferably in vitro synthesized, dsRNA targeting a plant MMR mRNA in combination with mutagenic nucleobases to produce a desired nucleotide alteration in the plant genome. As down regulation of transcript levels by dsRNA is transient, the MMR system will only be inactivated for a certain amount of time, preferably about 48-72 hrs. This window in time is usually sufficient as the mutagenic nucleobases are degraded rapidly in plant protoplasts and typically are eliminated after about 72 hours and therefore the TGA process preferably occurs within the 72 hours after introduction of the mutagenic nucleobase. After this period, the MMR transcripts will return to their normal levels thus preventing the accumulation of replication-associated mutations. This method is applicable to a wide range of plant species and is very flexible because transgenic lines expressing hairpin RNAi constructs do not have to be generated and screened for the desired down regulation, which is both time consuming and costly. In fact, EST's encoding components of the MMR system from many plant species are known (Table 1) and it has been found that these EST-sequences can serve as templates for the in vitro production of desired dsRNA.

### DETAILED DESCRIPTION OF THE INVENTION

The invention thus relates to a method for targeted gene alteration in plant cell protoplasts comprising transfecting the protoplasts with:
a dsRNA that targets plant MMR mRNA; and
a mutagenic nucleobase.

As discussed herein below, the transient down regulation of specific gene transcripts in plant protoplasts has been described earlier, but not regarding MMR transcripts in relation to TGA. The first studies were performed by Akashi et al. (2001 Antisense & Nucl. Acid Drug

Dev. 11: 359-367). They utilized so called hairpin RNAi constructs (plasmids) that consist of identical complementary regions of the target gene cloned as an inverted repeat and separated by a short non-specific DNA sequence. Upon transcription, these complementary regions of the target gene anneal to form a region of double stranded RNA with the non-specific DNA forming a loop structure. This double stranded RNA region is then processed into small interfering RNAs (siRNA) by DICER, which are then incorporated into the RISC complex and cause degradation of the target mRNA. The authors demonstrated that a plasmid expressing a hairpin RNAi targeting the GFP mRNA was able to suppress transient GFP expression in tobacco BY-2 cells. Therefore, it is not necessary to first integrate a hairpin RNAi construct into the plant genome to down regulate specific mRNA's. However, construction of plasmids containing hairpin RNAi constructs is difficult and time consuming, so other forms of mRNA inhibiting dsRNA were tested. In similar experiments, An et al. (2003 Biosci. Biotechnol. Biochem. 67: 2674-2677) prepared long double stranded RNA (dsRNA) by in vitro transcription targeting the luciferase mRNA. This was then co-transformed into Arabidopsis protoplasts together with a luciferase expressing plasmid and was shown to suppress transient luciferase activity. This suppression was independent of the length of the dsRNA used (50 bp, 100 bp, 250 bp or 500 bp) and a 90% inhibition luciferase expression was observed up to 14 days after protoplast transformation. Thus, a region of dsRNA prepared in vitro and transfected into the cell has been shown to give transient down regulation of specific mRNA's, but again, not for TGA and not for mRNA's associated with MMR. For practical application it is essential to demonstrate that in vitro prepared dsRNA can down regulate endogenous plant genes which, compared with transient GFP and luciferase expression, are expressed at relatively low levels. This has been demonstrated in two different plant species. Firstly, An et al. (2005 Biosci. Biotechnol. Biochem. 69: 415-418 showed that dsRNA could down regulate the mRNA of two endogenous Arabidopsis genes by 80% for three days at which point the mRNA levels returned to the control levels, presumably due to degradation of the dsRNA molecules. Secondly, Dubouzet et al. (2005 Biosci. Biotechnol. Biochem. 69: 63-70) showed similar results when using dsRNA to suppress mRNA's involved in the berberine biosynthetic pathway of Coptis japonica protoplasts.

In plant cells, dsRNA seems more suitable and are hence more preferred than other types of RNA for the transient suppression of endogenous gene transcripts than other types of RNA molecules (siRNA) more routinely used in animal studies. siRNA's are short (˜21 nt) single stranded RNA molecules that are synthesized in vitro and then transfected to the animal cells where they are directly incorporated into the RISC complex and direct the sequence specific cleavage of their target mRNA's. While siRNA's work efficiently in animal cells, their use in plant cells to suppress transcripts derived from endogenous plant genes has thus far not been described or suggested. Expression of siRNA's is sufficient to inhibit the accumulation of plant viruses in cultured plant cells (Vanitharani et al. 2003 Proc. Natl. Acad. Sci. USA 100: 9632-9636) or to reduce the transient expression of exogenously added GUS or luciferase genes (Bail et al. 2006 Plant Methods 2: 13) but there are no reports of siRNA being able to transiently suppress endogenous plant gene mRNA's. This suggests that endogenous plant mRNA can only be efficiently degraded when long dsRNA is used. In animal cells, dsRNA is not suitable for the suppression of endogenous mammalian gene transcripts. In mammalian cells dsRNA causes non-specific suppression and degradation of all mRNA species via the interferon pathway which is important as a defence system against viral infection and is triggered by viral dsRNA. Transfection of dsRNA to animal cells thus results in activation of this pathway and apoptosis. This pathway does not seem to be present in plant cells as transfection of dsRNA has not been reported to have any deleterious effect on protoplast survival. So, although the use of dsRNA in transfecting plant protoplasts has been demonstrated to work for certain specific genes, there is no indication or teaching that the MMR system is affected by the use of dsRNA that target the MMR-related mRNA's. In addition, all the studies cited above have demonstrated that the down regulation of plant mRNA's by dsRNA occurs when the protoplasts are derived from a plant cell suspension (an in vitro grown plant cell culture of undifferentiated cells). Such cultures are easy to use and provide an almost limitless source of plant cells. However, such cells cannot be compared with cells from mature plants. For example, unlike protoplasts derived from leaf mesophyll cells, tobacco BY-2 suspension cells divide much faster and are unable to regenerate into mature plants. Thus, at the outset of this study there was no indication that dsRNA would be able to down regulate an endogenous plant gene transcript in protoplasts derived from mesophyll cells, which must be used for the TGA process to allow eventual regeneration of mature plants.

The transfection with the dsRNA can be performed simultaneously, i.e. the dsRNA and the mutagenic nucleobase are added in one transfection step, which is preferred for efficiency reasons. However, in certain embodiments, it can be advantageous to transfect the protoplast first with the dsRNA, followed within a certain time by the mutagenic nucleobase or vice versa, i.e. first introduce the mutagenic nucleobase and later the dsRNA. In certain embodiments, this time period does not exceed 48 hours, preferably. In certain embodiments the transfection with the dsRNA and the mutagenic nucleobase (or vice versa) is spaced apart not more than 1, 2, 3, 4, 5, 6, 7, 8, 10, 12, 18, 24, 36, 48 hours. It can be advantageous to introduce the dsRNA first, to target the MMR genes, and when the MMR system is sufficiently down regulated, to introduce the mutagenic nucleobase. It can also be advantageous to introduce the mutagenic nucleobase first followed by the dsRNA as it may take some time before the MMR system is activated by the mutagenic nucleobase and the the window for successful TGA can be extended.

The dsRNA typically can have a length of from 30 to 5000 bp. A preferred length would be in the range of 100 to 500 bp

The MMR genes that can be targeted can in principle be any MMR-associated gene. There is a preference however, for known target genes of the MMR system, such as the MutS and/or MutL MMR genes, more preferably MSH2, MSH3, MSH6, MSH7, MLH1, MLH2, MLH3 and PMS1. In certain embodiments, one can determine the relevant genes by database analysis, identification of the genes that are by virtue of classification or identity related to MMR and test dsRNA for its activity. In certain embodiments, the dsRNA can be designed based on genes and gene fragments that have a close percentage identity to MMR associated genes such as those listed in Table 1. “Identity” is a measure of the identity of nucleotide sequences or amino acid sequences. In general, the sequences are aligned so that the highest order match is obtained. “Identity” per se has an art-recognized meaning and can be calculated using published techniques. See, e.g.: (COMPUTATIONAL MOLECULAR BIOLOGY, Lesk, A. M., ed., Oxford University Press, New York, 1988; BIOCOMPUTING: INFORMATICS AND GENOME PROJECTS, Smith, D. W., ed., Academic Press, New York, 1993; COMPUTER ANALYSIS OF SEQUENCE DATA, PART I, Griffin, A. M., and Griffin, H. G., eds., Humana Press, New Jersey, 1994; SEQUENCE ANALYSIS IN MOLECULAR BIOLOGY, von Heinje, G., Academic Press, 1987; and SEQUENCE ANALYSIS PRIMER; Gribskov, M. and Devereux, J., eds., M Stockton Press, New York, 1991). While there exist a number of methods to measure identity between two polynucleotide or polypeptide sequences, the term “identity” is well known to skilled artisans (Carillo, H., and Lipton, D., SIAM J. Applied Math (1988) 48:1073). Methods commonly employed to determine identity or similarity between two sequences include, but are not limited to, those disclosed in GUIDE TO HUGE COMPUTERS, Martin J. Bishop, ed., Academic Press, San Diego, 1994, and Carillo, H., and Lipton, D., SIAM J. Applied Math (1988) 48:1073. Methods to determine identity and similarity are codified in computer programs. Preferred computer program methods to determine identity and similarity between two sequences include, but are not limited to, GCS program package (Devereux, J., et al., Nucleic Acids Research (1984) 12(1):387), BLASTP, BLASTN, FASTA (Atschul, S. F. et al., J. Molec. Biol. (1990) 215:403).

As an illustration, by a polynucleotide having a nucleotide sequence having at least, for example, 95% “identity” to a reference nucleotide sequence encoding a polypeptide of a certain sequence it is intended that the nucleotide sequence of the polynucleotide is identical to the reference sequence except that the polynucleotide sequence may include up to five point mutations per each 100 nucleotides of the reference polypeptide sequence. In other words, to obtain a polynucleotide having a nucleotide sequence at least 95% identical to a reference nucleotide sequence, up to 5% of the nucleotides in the reference sequence may be deleted and/or substituted with another nucleotide, and/or a number of nucleotides up to 5% of the total nucleotides in the reference sequence may be inserted into the reference sequence. These mutations of the reference sequence may occur at the 5′ or 3′ terminal positions of the reference nucleotide sequence, or anywhere between those terminal positions, interspersed either individually among nucleotides in the reference sequence or in one or more contiguous groups within the reference sequence.

The method according to the present invention results in the down regulation of at least one or more MMR genes, preferably in plant cell protoplasts, sufficiently to allow TGA to be performed with the mutagenic nucleobase. Preferably the down regulation is specific, i.e. other mRNA s are not down regulated to an extent that the other biological systems operating the plant cell protoplast are significantly affected, i.e. are disturbed for not more than 5%, 10%, 15%, or 25% compared to their normal functionality, i.e. in absence of the dsRNA.

The plant can be any plant, and can be preferably selected from amongst monocots or dicots. Preferred plants are Cucurbitaceae, Gramineae, Solanaceae or Asteraceae (Compositae), maize/corn (Zea species), wheat (Triticum species), barley (e.g. Hordeum vulgare), oat (e.g. Avena sativa), sorghum (Sorghum bicolor), rye (Secale cereale), soybean (Glycine spp, e.g. G. max), cotton (Gossypium species, e.g. G. hirsutum, G. barbadense), Brassica spp. (e.g. B. napus, B. juncea, B. oleracea, B. rapa, etc), sunflower (Helianthus annus), safflower, yam, cassava, alfalfa (Medicago sativa), rice (Oryza species, e.g. O. sativa indica cultivar-group or japonica cultivar-group), forage grasses, pearl millet (Pennisetum spp. e.g. P. glaucum), tree species (Pinus, poplar, fir, plantain, etc), tea, coffea, oil palm, coconut, vegetable species, such as pea, zucchini, beans (e.g. Phaseolus species), hot pepper, cucumber, artichoke, asparagus, eggplant, broccoli, garlic, leek, lettuce, onion, radish, turnip, tomato, potato, Brussels sprouts, carrot, cauliflower, chicory, celery, spinach, endive, fennel, beet, fleshy fruit bearing plants (grapes, peaches, plums, strawberry, mango, apple, plum, cherry, apricot, banana, blackberry, blueberry, citrus, kiwi, figs, lemon, lime, nectarines, raspberry, watermelon, orange, grapefruit, etc.), ornamental species (e.g. Rose, Petunia, Chrysanthemum, Lily, Gerbera species), herbs (mint, parsley, basil, thyme, etc.), woody trees (e.g. species of Populus, Salix, Quercus, Eucalyptus), fibre species e.g. flax (Linum usitatissimum) and hemp (Cannabis sativa), and others.

Most preferred are Solanaceae, such as tobacco, tomato. Also preferred is lettuce and/or brassica.

The mutagenic nucleobase can be any mutagenic nucleobase as described in the art such as those disclosed in the applicants applications WO2007073149, WO2007073154 and WO2007073170.

Thus, the mutagenic nucleobase may comprise one or more of:

a. phosphorothioate modifications, preferably near or at one or both ends of the mutagenic nucleobase;

b. propyne substitutions, preferably not near or at one or both ends of the mutagenic nucleobase

c. LNA substitutions, preferably not near or at one or both ends of the mutagenic nucleobase

The phosphorothioate modifications may serve to protect the nucleobase from nucleases present in the protoplast system.

The propyne substitutions that are preferably not near or at one or both ends of the mutagenic nucleobase may exert an enhanced binding affinity with the target sequence to be altered by TGA. The LNA substitutions that are preferably not near or at one or both ends of the mutagenic nucleobase may also exert an enhanced binding affinity with the sequence to be altered by TGA. The use of LNA or propyne modified oligonucleotides may lead to increased efficiencies of TGA.

The modified mutagenic nucleobases that can be used are described further in more detail herein below.

LNA modified mutagenic nucleobases:

In certain embodiments, the mutagenic nucleobase comprises at least one, preferably at least 2, more preferably at least 3 LNA modified nucleotide(s). In certain embodiments, the mutagenic nucleobase can contain more than 1, 2, 3, 4, 5, 6, 7, 8, 9, or 10 LNA modified nucleotides. In certain embodiments, the mutagenic nucleobase can contain up tol, 2, 3, 4, 5, 6, 7, 8, 9, or 10 LNA modified nucleotides. In certain embodiments, the mutagenic nucleobase can comprise ranges of LNA that can be comprised of the above upper and lower limits

In certain embodiments, the at least one LNA is positioned at a distance of at most 10 nucleotides, preferably at most 8 nucleotides, more preferably at most 6 nucleotides, even more preferably at most 4, 3, or 2 nucleotides from the mismatch. In a more preferred embodiment the at least one LNA is positioned at a distance of 1 nucleotide from the mismatch, i.e. one nucleotide is positioned between the mismatch and the LNA. In certain embodiments relating to mutagenic nucleobases containing more than one LNA, each LNA is located at a distance of at least one nucleotides from the mismatch. In a preferred embodiment, LNAs are not located adjacent to each other but are spaced apart by at least one nucleotide, preferably two or three nucleotides. In certain embodiments, in the case of two or more (even numbers of) LNA modifications of the mutagenic nucleobase, the modifications are spaced at (about) an equal distance from the mismatch. In other words, preferably the LNA modifications are positioned symmetrically around the mismatch. For example, in a preferred embodiment, two LNAs are positioned symmetrically around the mismatch at a distance of 1 nucleotide from the mismatch (and 3 nucleotides from each other). In certain embodiments, the LNAs are located starting from a position located 4-6 nucleotides from the ends of the mutagenic nucleobase, independently at either end

In certain embodiments, at most 50% of the modified nucleotides of the mutagenic nucleobase are LNA derivatives, i.e. the conventional A, T, C, or G is replaced by its LNA counterpart, preferably at most 40%, more preferably at most 30%, even more preferably at most 20%, and most preferably at most 10%. Locked Nucleic Acid (LNA) is a DNA analogue with very interesting properties for use in antisense gene therapy. LNAs are bicyclic and tricyclic nucleoside and nucleotide analogues and the mutagenic nucleobases that contain such analogues. The basic structural and functional characteristics of LNAs and related analogues are disclosed in various publications and patents, including WO 99/14226, WO 00/56748, WO00/66604, WO 98/39352, U.S. Pat. Nos. 6,043,060, and 6,268,490, all of which are incorporated herein by reference in their entireties.

Specifically, it combines the ability to discriminate between correct and incorrect targets (high specificity) with very high bio-stability (low turnover) and unprecedented affinity (very high binding strength to target). In fact, the affinity increase recorded with LNA leaves the affinities of all previously reported analogues in the low-to-modest range.

LNA is an RNA analogue, in which the ribose is structurally constrained by a methylene bridge between the 2′-oxygen and the 4′-carbon atoms. This bridge restricts the flexibility of the ribofuranose ring and locks the structure into a rigid bicyclic formation. This so-called N-type (or 3′-endo) conformation results in an increase in the Tm of LNA containing duplexes, and consequently higher binding affinities and higher specificities. NMR spectral studies have actually demonstrated the locked N-type conformation of the LNA sugar, but also revealed that LNA monomers are able to twist their unmodified neighbour nucleotides towards an N-type conformation. Importantly, the favourable characteristics of LNA do not come at the expense of other important properties as is often observed with nucleic acid analogues.

LNA can be mixed freely with all other chemistries that make up the DNA analogue universe. LNA bases can be incorporated into mutagenic nucleobases as short all-LNA sequences or as longer LNA/DNA chimeras. LNAs can be placed in internal, 3′ or 5′-positions. However, due to their rigid bicyclic conformations, LNA residues sometimes disturb the helical twist of nucleic acid strands. It is hence generally less preferred to design a mutagenic nucleobase with two or more adjacent LNA residues. Preferably, the LNA residues are separated by at least one (modified) nucleotide that does not disturb the helical twist, such as a conventional nucleotide (A, C, T, or G).

The originally developed and preferred LNA monomer (the β-D-oxy-LNA monomer) has been modified into new LNA monomers. The novel α-L-oxy-LNA shows superior stability against 3′ exonuclease activity, and is also more powerful and more versatile than β-D-oxy-LNA in designing potent antisense oligonucleotides. Also xylo-LNAs and L-ribo LNAs can be used, as disclosed in WO9914226, WO00/56748, WO00/66604. In the present invention, any LNA of the above types is effective in achieving the goals of the invention, i.e. improved efficiency of TGA, with a preference for β-D-LNA analogues.

In the art on TGA, LNA modification has been listed amongst a list of possible mutagenic nucleobase modifications as alternatives for the chimeric molecules used in TGA. However, there is no indication in the art thus far that suggests that LNA modified single-stranded mutagenic nucleobase enhances TGA efficiency significantly to the extent that has presently been found when the LNA is positioned at least one nucleotide away from the mismatch and/or the mutagenic nucleobase does not contain more than about 75% (rounded to the nearest whole number of nucleotides) LNAs.

Propynyl modified mutagenic nucleobases:

Mutagenic nucleobases containing pyrimidine nucleotides with a propynyl group at the C5 position form more stable duplexes and triplexes than their corresponding pyrimidine derivatives. Purine with the same propyne substituent at the 7-position form even more stable duplexes and are hence preferred. In certain preferred embodiments, efficiency was further increased through the use of 7-propynyl purine nucleotides (7-propynyl derivatives of 8-aza-7-deaza-2′-deoxyguanosine and 8-aza-7-deaza-2′-deoxyadenine) which enhance binding affinity to an even greater degree than C5-propyne pyrimidine nucleotides. Such nucleotides are disclosed inter alia in He & Seela, 2002 Nucleic Acids Res. 30: 5485-5496.

A propynyl group is a three carbon chain with a triple bond. The triple bond is covalently bound to the nucleotide basicstructure which is located at the C5 position of the pyrimidine and at the 7-postion of the purine nucleotide . Both cytosine and thymidine can be equipped with C5-propynyl group, resulting in C5-propynyl-cytosine and C5-propynyl-thymidine, respectively. A single C5-propynyl-cytosine residue increases the Tm by 2.8° C., a single C5-propynyl-thymidine by 1.7° C.

(Froehler et al. 1993 Tetrahedron Letters 34: 1003-6; Lacroix et al. 1999 Biochemistry 38: 1893-1901; Ahmadian et al. 1998 Nucleic Acids Res. 26: 3127-3135; Colocci et al. 1994 J. Am. Chem. Soc 116: 785-786). This is attributed to the hydrophobic nature of 1-propyne groups at the C5 position and it also allows better stacking of the bases since the propyne group is planar with respect to the heterocyclic base. In certain embodiments, the modified nucleobase is a propyne modified nucleobase, most preferably a C7-propyne purine or C5-propyne pyrimidine. In certain embodiments, the purine is adenosine or guanosine and/or the pyrimidine is cytosine, uracil or thymidine, more prefereably the modified nucleotide is a pyrimidine and/or the modified nucleotide is a purine.

In certain embodiments , at least 10% of the pyrimidines and/or purines are replaced by their respective propynylated derivatives, preferably at least 50%, more preferably at least 75% and most preferably at least 90%

Preferably the nucleotide at the position of the mismatch is not modified. In one embodiment, the at least one modified nucleotide is not located adjacent to the mismatch, and preferably is located within 2, 3, 4, 6, 7, 8, 9, or 10 nucleotides of the mismatch.

The mutagenic nucleobases according to the invention may contain further modifications to improve the hybridisation characteristics such that the mutagenic nucleobase exhibits increased affinity for the target DNA strand so that intercalation of the mutagenic nucleobases is easier. The mutagenic nucleobases can also be further modified to become more resistant against nucleases, to stabilise the triplex or quadruplex structure. Modification of the C5 propyne substituted pyrimidine mutagenic nucleobases can comprise phosphorothioate modification, 2-OMe substitutions, the use of different LNAs (Locked nucleic acids), PNAs (Peptide nucleic acids), ribonucleotide and other bases that modifies, preferably enhances, the stability of the hybrid between the mutagenic nucleobases and the acceptor strand.

The method according to the invention finds application in altering a cell, correcting a mutation by restoration to wild type, inducing a mutation, inactivating an enzyme by disruption of coding region, modifying bioactivity of an enzyme by altering coding region, modifying a protein by disrupting the coding region, mismatch repair, targeted alteration of (plant)genetic material, including gene mutation, targeted gene repair and gene knockout.

### BRIEF DESCRIPTION OF THE FIGURES

FIG. 1 The regions of the tobacco and tomato PMS1 regions used as a template for dsRNA production were translated and aligned with other PMS1 orthologs

FIG. 2 Relative PMS1 transcript levels in tobacco mesophyll protoplasts after introduction of dsRNA targeting PMS1 transcripts. Mesophyll protoplasts were treated with dsRNA (RNA) or water (MQ) and total RNA was isolated from the protoplasts directly (RNA-0, MQ-0), 24 hours (RNA-1, MQ-1), 48 hours (RNA-2, MQ-2), 72 hours (RNA-3, MQ-3) after transfection.

FIG. 3 Sequence of the tomato MLH1 and MSH2 cDNA's. The PCR product produced for dsRNA production is indicated.

### EXAMPLES

### Example 1

### Transient Suppression of PMS1 mRNA in Tobacco Mesophyll Protoplasts

Experiments were performed to demonstrate that dsRNA is able to downregulate the PMS1 mRNA in tobacco mesophyll protoplasts.

### Materials and Methods

Generation of dsRNA

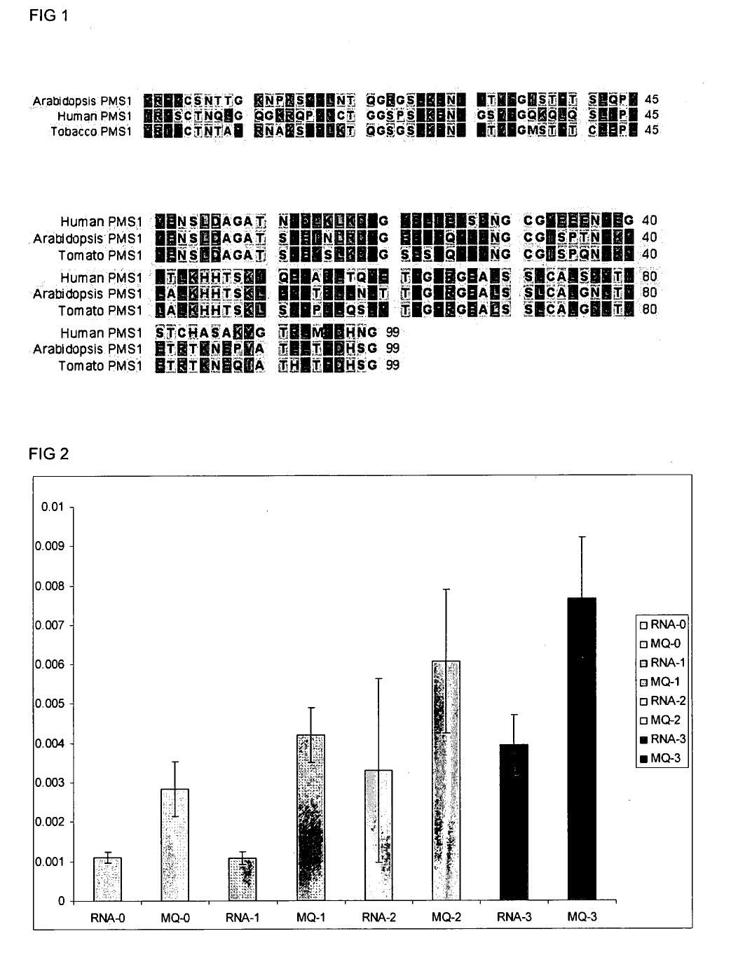

The public databases were screened for tobacco orthologs of Arabidopsis PMS1. Using RACE PCR the full length clones were then isolated and sequenced. Primers were designed to amplify a PCR product of tobacco PMS1 that would serve as a template for RNA synthesis. This resulted in a PCR product of 186 bps. Translation of the PCR product and its alignment with other PMS1 orthologs is shown in FIG. 1.

Per template, 2 PCR products were amplified which were identical in sequence but had a T7 RNA polymerase promoter sequence on opposite strands. 1 μg of each PCR product was used for in vitro RNA transcription using the T7 RiboMAX Express RNAi System (Promega) which resulted in the production of single stranded RNA corresponding to either the upper of lower strand of the PCR products. Complementary RNA strands were purified and annealed to generate dsRNA as per the manufacturers instructions.

Tobacco Protoplast Isolation and Transformation

In vitro shoot cultures of Nicotiana tabacum cv Petit Havana line SR1 are maintained on MS20 medium with 0.8% Difco agar in high glass jars at 16/8 h photoperiod of 2000 lux at 25° C. and 60-70% RH. MS20 medium is basic Murashige and Skoog's medium (Murashige, T. and Skoog, F., Physiologia Plantarum, 15: 473-497, 1962) containing 2% (w/v) sucrose, no added hormones and 0.8% Difco agar. Fully expanded leaves of 3-6 week old shoot cultures are harvested. The leaves are sliced into 1 mm thin strips, which are then transferred to large (100 mm×100 mm) Petri dishes containing 45 ml MDE basal medium for a preplasmolysis treatment of 30 min. MDE basal medium contained 0.25 g KCl, 1.0 g MgSO4.7H2O, 0.136 g of KH2PO4, 2.5 g polyvinylpyrrolidone (MW 10,000), 6 mg naphthalene acetic acid and 2 mg 6-benzylaminopurine in a total volume of 900 ml. The osmolality of the solution is adjusted to 600 mOsm.kg-1 with sorbitol, the pH to 5.7. 5 mL of enzyme stock SR1 are then added. The enzyme stock consists of 750 mg Cellulase Onozuka R10, 500 mg driselase and 250 mg macerozyme R10 per 100 ml, filtered over Whatman paper and filter-sterilized. Digestion is allowed to proceed overnight in the dark at 25° C. The digested leaves are filtered through 50 μm nylon sieves into a sterile beaker. An equal volume of cold KCl wash medium is used to wash the sieve and pooled with the protoplast suspension. KCl wash medium consisted of 2.0 g CaCl2.2H2O per liter and a sufficient quantity of mannitol to bring the osmolality to 540 mOsm.kg-1. The suspension is transferred to 10 mL tubes and protoplasts are pelleted for 10 min at 85×g at 4° C. The supernatant is discarded and the protoplast pellets carefully resuspended into 5 mL cold MLm wash medium, which is the macro-nutrients of MS medium (Murashige, T. and Skoog, F., Physiologia Plantarum, 15: 473-497, 1962) at half the normal concentration, 2.2 g of CaCl2.2H2O per liter and a quantity of mannitol to bring the osmolality to 540 mOsm.kg-1. The content of 2 tubes is combined and centrifuged for 10 min at 85×g at 4° C. The supernatant is discarded and the protoplast pellets carefully resuspended into 5 mL cold MLs wash medium which is MLm medium with mannitol replaced by sucrose.

The content of 2 tubes is pooled and 1 mL of KCl wash medium added above the sucrose solution care being taken not to disturb the lower phase. Protoplasts are centrifuged for 10 min at 85×g at 4° C. The interphase between the sucrose and the KCl solutions containing the live protoplasts is carefully collected. An equal volume of KCl wash medium is added and carefully mixed. The protoplast density is measured with a haemocytometer.

Introduction of dsRNA and Protoplast Regeneration

The protoplast suspension is centrifuged at 85×g for 10 minutes at 5° C. The supernatant is discarded and the protoplast pellet resuspended to a final concentration of 106.mL-1 in KCl wash medium. In a 10 mL tube, 250 μL of protoplast suspension +/−12.5 μg dsRNA and 250 μl of PEG solution (40% PEG4000 (Fluka #81240), 0.1M Ca(NO3)2, 0.4M mannitol) are gently but thoroughly mixed. After 20 min. incubation at room temperature, 5 mL cold 0.275 M Ca(NO3)2 is added dropwise. The protoplast suspension is centrifuged for 10 min at 85×g at 4° C. and the supernatant discarded. The protoplast pellet was then carefully resuspended in 2.5 mL To culture medium supplemented with 50 μg.mL-1 cefotaxime and 50 μg.mL-1 vancomycin. TO culture medium contained (per liter, pH 5.7) 950 mg KNO3, 825 mg NH4NO3, 220 mg CaCl2.2H2O, 185 mg MgSO4.7H2O, 85 mg KH2PO4, 27.85 mg FeSO4.7H2O, 37.25 mg Na2EDTA.2H2O, the micro-nutrients according to Heller's medium (Heller, R., Ann Sci Nat Bot Biol Veg 14: 1-223, 1953), vitamins according to Morel and Wetmore's medium (Morel, G. and R. H. Wetmore, Amer. J. Bot. 38: 138-40, 1951), 2% (w/v) sucrose, 3 mg naphthalene acetic acid, 1 mg 6-benzylaminopurine and a quantity of mannitol to bring the osmolality to 540 mOsm.kg-1 and transferred to a 35 mm Petri dish.

Quantification of PMS1 mRNA levels

Total RNA was isolated from protoplasts using the RNAeasy Kit (Qiagen). cDNA sysnthesis was performed using the Quantitect RT kit (Qiagen). Levels of endogenous PMS1 were measured using using a Light Cycler apparatus (Roche) and the primers 5′-AGCAGTTCCCTTCAGCAAAAAT [SEQ ID NO 1] and 5′-GAATCGGCGGTATCATCCTTAT [SEQ ID NO 2] amplifying a 126 bp product derived from the tobacco PMS1 mRNA. For each time point, 5 independent protoplast transfections were performed. For normalization of tobacco PMS1, the levels of actin mRNA were measured in each sample.

Results

The results of the qPCR analysis are shown in FIG. 1.

In tobacco mesophyll protoplasts, PMS1 mRNA levels can be significantly reduced by addition of dsRNA. The results demonstrate that 24 hours after transfection of the dsRNA, the PMS1 mRNA level drops to 25% of the control level. The PMS1 mRNA down regulation is clearly transient, as a partial recovery of the PMS1 mRNA levels was observed after 48-72 hours, presumably due to degradation of the dsRNA. The dsRNA had no aspecific effects on the expression of other mRNA species, such as the level of actin mRNA, assessed in each sample to normalize the PMS1 expression. Thus, in vitro synthesized dsRNA is able to transiently and specifically down regulate an MMR mRNA in tobacco mesophyll protoplasts.

### Example 2

### TGA Experiments in Tobacco Using dsRNA Targeted to PMS1

Experiments were performed using the mutagenic nucleobase PB124 (5′A*T*C*A*TCCTACGTTGCACTTG*A*C*C*G [SEQ ID NO 3]). It corresponds to the non-transcribed strand of the SurB gene from tobacco that encodes an ortholog of acetolactate synthase (ALS). The oligonucleotide contains a single mismatch with SurB (underlined) that drives the SurB Proline 191 to glutamic acid conversion, conferring a dominant resistance phenotype to the sulfonylurea type herbicides. The asterisks represent phosphorothioate linkages in which a non-bridging oxygen atom in the phosphate linkage is substituted by a sulphur atom. Such modified linkages are known to be more resistant to exonuclease attack and thus prolong the lifetime of the mutagenic nucleobase in the cell.

Tobacco protoplasts were prepared as described in Example 1. 12.5 μg ds RNA and 10 μg PB124 were transfected to an aliquot of protoplasts and which were finally resuspended in 1.25 ml of T0 culture medium. The suspension was transferred to a 35 mm Petri dish. An equal volume of T0 agarose medium is added and gently mixed. Samples were incubated at 25° C. in the dark and further cultivated as described below.

Protoplast Cultivation and Regeneration

After 10 days of cultivation, the agarose slab is cut into 6 equal parts and transferred to a Petri dish containing 22.5 mL MAP1AO medium supplemented with 20 nM chlorsulfuron. This medium consisted of (per liter, pH 5.7) 950 mg KNO3, 825 mg NH4NO3, 220 mg CaCl2.2H2O, 185 mg MgSO4.7H2O, 85 mg KH2PO4, 27.85 mg FeSO4.7H20, 37.25 mg Na2EDTA.2H2O, the micro-nutrients according to Murashige and Skoog's medium (Murashige, T. and Skoog, F., Physiologia Plantarum, 15: 473-497, 1962) at one tenth of the original concentration, vitamins according to Morel and Wetmore's medium (Morel, G. and R. H. Wetmore, Amer. J. Bot. 38: 138-40, 1951), 6 mg pyruvate, 12 mg each of malic acid, fumaric acid and citric acid, 3% (w/v) sucrose, 6% (w/v) mannitol, 0.03 mg naphthalene acetic acid and 0.1 mg 6-benzylaminopurine. Samples are incubated at 25° C. in low light for 6-8 weeks. Growing calli are then transferred to MAP1 medium and allowed to develop for another 2-3 weeks. MAP1 medium has the same composition as MAP1AO medium, with however 3% (w/v) mannitol instead of 6%, and 46.2 mg.l-1 histidine (pH 5.7). It was solidified with 0.8% (w/v) Difco agar.

Calli are then transferred to RP medium using sterile forceps. RP medium consisted of (per liter, pH 5.7) 273 mg KNO3, 416 mg Ca(NO3)2.4H2O, 392 mg Mg(NO3)2.6H2O, 57 mg MgSO4.7H2O, 233 mg (NH4)2SO4, 271 mg KH2PO4, 27.85 mg FeSO4.7H2O, 37.25 mg Na2EDTA.2H2O, the micro-nutrients according to Murashige and Skoog's medium at one fifth of the published concentration, vitamins according to Morel and Wetmore's medium (Morel, G. and R. H. Wetmore, Amer. J. Bot. 38: 138-40, 1951), 0.05% (w/v) sucrose, 1.8% (w/v) mannitol, 0.25 mg zeatin and 41 nM chlorsulfuron, and is solidified with 0.8% (w/v) Difco agar. Mature shoots are transferred to rooting medium after 2-3 weeks.

### Results

The results of the TGA experiments are shown in Table 3.

TABLE 3
TGA experiments in tobacco protoplasts using dsRNA to down regulate
endogenous PMS1 expression

| | Mutagenic | # chlorsulfuron resistant calli |
|---|---|---|
| dsRNA | nucleobase | per 1 × 10⁶ protoplasts |
| − | − | 0 |
| − | + | 6 |
| + | − | 0 |
| + | + | 192 |

Down regulation of tobacco PMS1 increases the TGA efficiency at least 30 fold. Shoots were regenerated from 20 calli treated with mutagenic nucleobase and dsRNA and genotyped. DNA was isolated from these plants and SurB PCR products including the P191 were amplified and sequenced. All plants showed the expected P191Q mutation and so we conclude that TGA had indeed occurred in these lines.

### TGA Experiments in Tomato Using dsRNA Targeted to PMS1

These experiments were performed using the mutagenic nucleobases listed in Table 4. In tomato ALS is a multicopy gene, two full length EST's are present in the Plant Transcript Database (http://planta.tigr.org). In our study we have defined transcript TA37274_{—}4081 as ALS1 and transcript TA37275_{—}4081 as ALS2. ALS1 encodes a protein of 659AA while ALS2 encodes a protein of 657AA. ALS1 and ALS2 show 93% and 96% identity at the DNA and protein levels respectively. The two proteins mainly differ in the signal peptide regions of the proteins responsible for chloroplast targeting. Despite these differences, both ALS1 and ALS2 proteins are both predicted to be targeted to the chloroplast. Previous studies have shown that several amino acid changes at conserved residues of ALS are sufficient to confer a semi-dominant resistance to the sulfonylurea class of herbicides. One of these is the P184Q change. We have previously found that the TGA efficiency in tomato protoplasts is enhanced 8 fold when C5-propyne and LNA (locked nucleic acid) modifications are included on the mutagenic nucleobase. Thus, in this study we have introduced normal DNA mutagenic nucleobases or C5-propyne and LNA modified mutagenic nucleobases designed to produce a P184Q alteration in ALS2 simultaneously with dsRNA (205 bps) targeting tomato PMS1 into tomato leaf protoplasts.

TABLE 4

| SEQ ID | Oligo | Sequence | Mutation |
|---|---|---|---|
| 4 | 95 | A*T*C*A*TCCTCCTCTGCACTTG*A*C*C*G | P184Q |
| 5 | 44 | A*z*y*A*zyyzyyvyTGwAyzzG*A*y*y*G | P184Q |
| 6 | 80 | G*z*A*y*GzAyAxzCAxzAyGzA*G*G*A*U | None |

y = 5-propynyl-2′-deoxycytidine; z = 5-propynyl-2′-deoxyuracil; s = LNA A; v = LNA T; w = LNA C; x = LNA G; U = deoxyuracil; * = phosphorothioate linkages.
All mutagenic nucleobases were synthesized by Eurogentec and HPLC purified.
The nucleotide that forms a mismatch with the target is underlined.

Tomato Protoplast Experiments

Tomato Protoplast Isolation and Purification

Isolation and regeneration of tomato leaf protoplasts has been previously described (Shahin, 1985 Theor. Appl. Genet. 69: 235-240; Tan et al. 1987 Theor. Appl. Genet. 75: 105-108; Tan et al. 1987 Plant Cell Rep. 6: 172-175) and the solutions required can be found in these publications. Briefly, Solanum lycopersicum seeds were sterilized with 0.1% hypochlorite grown in vitro on sterile MS20 medium in a photoperiod of 16/8 hours at 2000 lux at 25° C. and 50-70% relative humidity. 1 g of freshly harvested leaves were placed in a dish with 5 ml CPW9M and, using a scalpel blade, cut perpendicular to the main stem every mm. These were transferred a fresh plate of 25 ml enzyme solution (CPW9M containing 2% cellulose onozuka RS, 0.4% macerozyme onozuka R10, 2.4-D (2 mg/ml), NAA (2 mg/ml), BAP (2 mg/ml) pH5.8) and digestion proceeded overnight at 25° C. in the dark. The protoplasts were then freed by placing them on an orbital shaker (40-50 rpm) for 1 hour. Protoplasts were separated from cellular debris by passing them through a 50 μm sieve, and washing the sieve 2× with CPW9M. Protoplasts were centrifuged at 85 g, the supernatant discarded, and then taken up in half the volume of CPW9M. Protoplasts were finally taken up in 3 ml CPW9M and 3 ml CPW18S was then added carefully to avoid mixing the two solutions. The protoplasts were spun at 85 g for 10 mins and the viable protoplasts floating at the interphase layer were collected using a long Pasteur pipette. The protoplast volume was increased to 10 ml bp adding CPW9M and the number of recovered protoplasts was determined in a haemocytometer. The protoplast suspension is centrifuged at 85 g for 10 minutes at 5° C. The supernatant is discarded and the protoplast pellet resuspended to a final concentration of 10⁶.mL-1 in CPW9M wash medium. In a 10 mL tube, 250 μL of protoplast suspension +/−12.5 μg dsRNA and 250 μl of PEG solution (40% PEG4000 (Fluka #81240), 0.1M Ca(NO3)2, 0.4M mannitol) are gently but thoroughly mixed. After 20 min. incubation at room temperature, 5 mL cold 0.275 M Ca(NO3)2 is added dropwise. The protoplast suspension is centrifuged for 10 min at 85×g at 4° C. and the supernatant discarded.

Tomato protoplasts were embedded in alginate solution for regeneration and selection of herbicide resistant calli. 2 ml of alginate solution was added (mannitol 90 g/l, CaCl2.2H2O 140 mg/l, alginate-Na 20 g/l (Sigma A0602)) and was mixed thoroughly by inversion. 1 ml of this was layered evenly on a Ca-agar plate (72.5 g/l mannitol, 7.35 g/l CaCl2.2H2O, 8 g/l agar) and allowed to polymerize. The alginate discs were then transferred to 4 cm Petri dishes containing 4 ml of K8p culture medium and incubated for 7 days in the dark at 30° C. without herbicide selection. Discs were then cut up into 5 mm broad strips and layered on TM-DB callus induction medium containing 20 nM chlorsulfuron. Herbicide resistant calli appeared after 4-5 weeks incubation at 30° C., and individuals were then transferred to GM-ZG shooting medium containing 20 nM chlorsulfuron for further growth.

### Results

The results of the TGA experiments using dsRNA to down regulate PMS1 mRNA levels are shown in Table 5.

TABLE 5
dsRNA targeting PMS1 enhances the TGA efficiency
in tomato mesophyll protoplasts

| Mutagenic | | # chlorsulfuron resistant calli |
|---|---|---|
| nucleobase | dsRNA | per 1 × 10⁶ protoplasts |
| 95 | − | 1 |
| 95 | + | 37 |
| 44 | − | 5 |
| 44 | + | 186 |
| 80 | − | 0 |
| 80 | + | 0 |

As in tobacco, transient down regulation of PMS1 by dsRNA in tomato enhances the TGA frequency approximately 30 fold. Analysis of the region of the ALS2 gene containing the P184 codon demonstrated that the herbicide resistant calli did indeed have the expected P184Q alteration.

### Example 3

### Suppression of MMR mRNA's in Tomato Mesophyll Protoplasts by dsRNA

Experiments were performed to demonstrate that dsRNA is able to down regulate the MLH1 mRNA in tomato mesophyll protoplasts.

Generation of Tomato MLH1 dsRNA

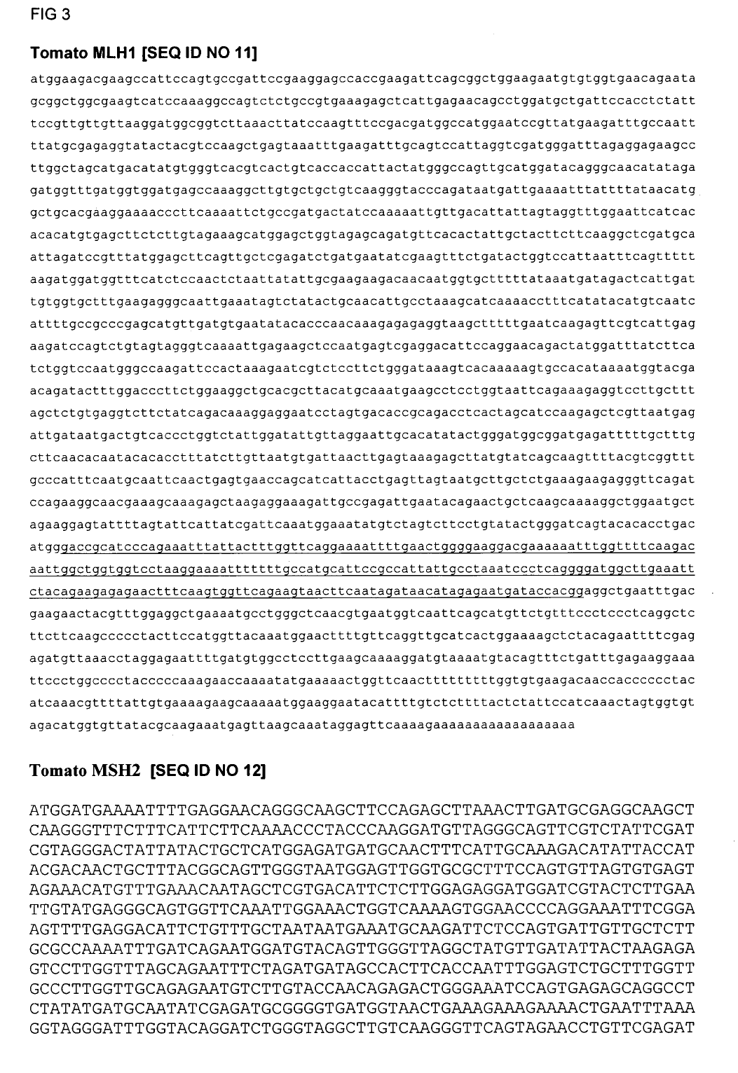

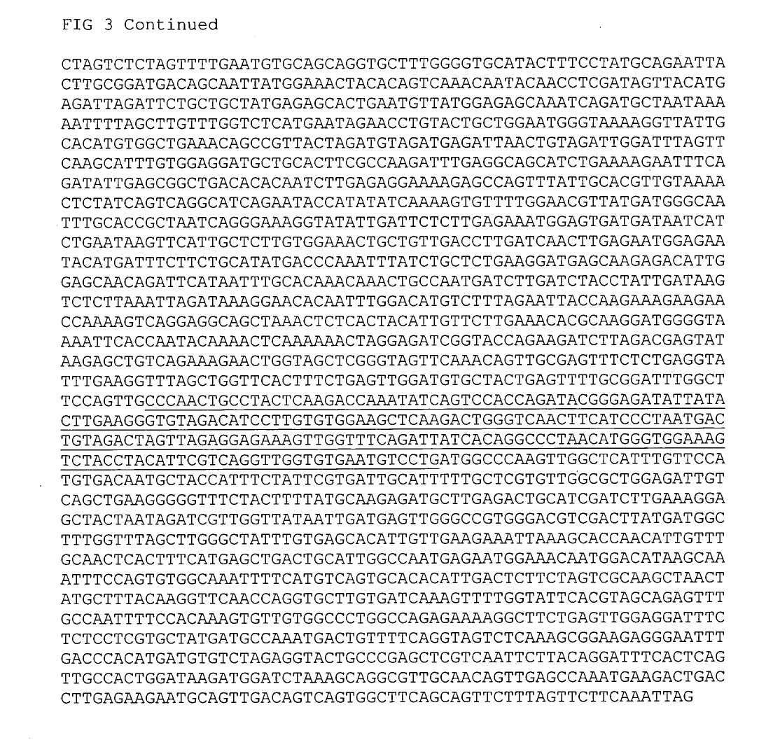

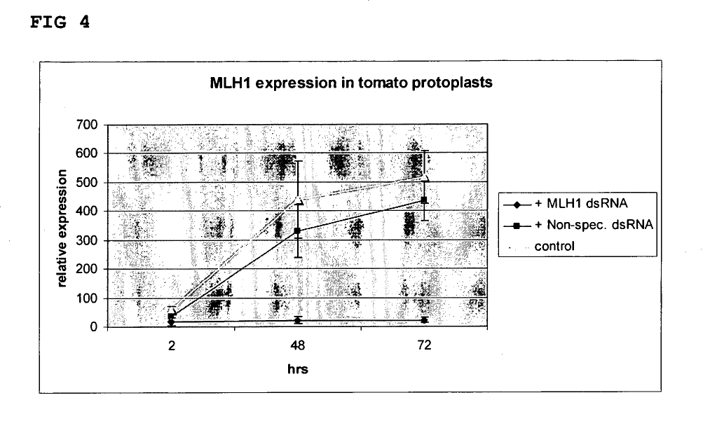

The public tomato genome databases were screened for tomato orthologs of Arabidopsis MLH1 and MSH2. Primers were designed to amplify fragments of these genes that would serve as a template for RNA synthesis. The PCR product were produced using tomato cDNA as a template. The sequence of tomato MLH1 and MSH2 and the regions used for dsRNA synthesis is shown (underlined) in FIG. 3.

Per template; 2 PCR products were amplified which were identical in sequence but had a T7 RNA polymerase promoter sequence on opposite strands. 1 μg of each PCR product was used for in vitro RNA transcription using the T7 RiboMAX Express RNAi System (Promega) which resulted in the production of single stranded RNA corresponding to either the upper or lower strand of the PCR products. Complementary RNA strands were purified and annealed to generate dsRNA as per the manufacturers instructions. In addition, we also produced a dsRNA molecule of identical length but comprised of a random DNA sequence (non-specific dsRNA). This was included in the experiments as an extra control to establish if dsRNA affected mRNA abundance in a non-specific manner.

Tomato Protoplasts

Tomato Protoplast Isolation and Purification

Isolation and regeneration of tomato leaf protoplasts has been previously described (Shahin, 1985 Theor. Appl. Genet. 69: 235-240; Tan et al. 1987 Theor. Appl. Genet. 75: 105-108; Tan et al. 1987 Plant Cell Rep. 6: 172-175) and the solutions required can be found in these publications. Briefly, Solanum lycopersicum seeds were sterilized with 0.1% hypochlorite grown in vitro on sterile MS20 medium in a photoperiod of 16/8 hours at 2000 lux at 25° C. and 50-70% relative humidity. 1 g of freshly harvested leaves were placed in a dish with 5 ml CPW9M and, using a scalpel blade, cut perpendicular to the main stem every mm. These were transferred a fresh plate of 25 ml enzyme solution (CPW9M containing 2% cellulose onozuka RS, 0.4% macerozyme onozuka R10, 2.4-D (2 mg/ml), NAA (2 mg/l), BAP (2 mg/ml) pH5.8) and digestion proceeded overnight at 25° C. in the dark. The protoplasts were then freed by placing them on an orbital shaker (40-50 rpm) for 1 hour. Protoplasts were separated from cellular debris by passing them through a 50 μm sieve, and washing the sieve 2× with CPW9M. Protoplasts were centrifuged at 85 g, the supernatant discarded, and then taken up in half the volume of CPW9M. Protoplasts were finally taken up in 3 ml CPW9M and 3 ml CPW18S was then added carefully to avoid mixing the two solutions. The protoplasts were spun at 85 g for 10 mins and the viable protoplasts floating at the interphase layer were collected using a long pasteur pipette. The protoplast volume was increased to 10 ml by adding CPW9M and the number of recovered protoplasts was determined in a haemocytometer. The protoplast suspension is centrifuged at 85×g for 10 minutes at 5° C. The supernatant is discarded and the protoplast pellet resuspended to a final concentration of 10⁶.mL-1 in CPW9M wash medium. In a 10 mL tube, 250 μL of protoplast suspension +/−12.5 μg dsRNA and 250 μl of PEG solution (40% PEG4000 (Fluka #81240), 0.1M Ca(NO3)2, 0.4M mannitol) are gently but thoroughly mixed. After 20 min. incubation at room temperature, 5 mL cold 0.275 M Ca(NO3)2 is added dropwise. The protoplast suspension is centrifuged for 10 min at 85×g at 4° C. and the supernatant discarded.

Tomato protoplasts were embedded in alginate solution for regeneration and selection of herbicide resistant calli. 2 ml of alginate solution was added (mannitol 90g/l, CaCl2.2H2O 140 mg/l, alginate-Na 20 g/l (Sigma A0602)) and was mixed thoroughly by inversion. 1 ml of this was layered evenly on a Ca-agar plate (72.5 g/l mannitol, 7.35 g/l CaCl2.2H2O, 8 g/l agar) and allowed to polymerize. The alginate discs were then transferred to 4 cm Petri dishes containing 4 ml of K8p culture medium. Protoplasts were freed from the alginate by incubation of the discs in a sodium citrate solution and subsequently harvested.

Quantification of MLH1 mRNA Levels

Total RNA was isolated from protoplasts using the RNAeasy Kit (Qiagen). cDNA synthesis was performed using the Quantitect RT kit (Qiagen). Levels of endogenous MLH1 were measured using using a Light Cycler apparatus (Roche) and the primers 5′-CCTGGTCTATTGGATATTGTTAG [SEQ ID NO 7] and 5′- GCTTGAGCAGTTCTGTATTC [SEQ ID NO 8], amplifying a 302 bps product derived from the tomato MLH1 mRNA. For each time point, 3 independent protoplast transfections were performed and the qPCR reactions were performed in triplicate. For normalization of tomato MLH1 levels, the levels of the tomato GAPDH mRNA were measured in each sample using the following primers, 5′-GCAATCAAGGAGGAATCAGAGG [SEQ ID No 9] and 5′-CCAGCAGCATCAATCAAGCC [SEQ ID No 10].

### Results

The results of the qPCR analysis are shown in FIG. 4. In tomato mesophyll protoplasts, MLH1 mRNA levels can be significantly reduced by addition of dsRNA. The MLH1 mRNA levels increase rapidly after protoplast isolation in the control samples, but this is not the case in the protoplasts treated with the MLH1 dsRNA where no increase in the levels is observed. None of the dsRNA species had an effects on the expression of other mRNA species, such as the level of GAPDH mRNA, assessed in each sample to normalize the MLH1 expression. Thus, in vitro synthesized dsRNA is able to transiently and specifically down regulate an MMR mRNA in tomato mesophyll protoplasts. We observed similar effects on the tomato MSH2 mRNA when protoplasts were transfected with MSH2 dsRNA.

### Example 4

### TGA Experiments in Tobacco Protoplast Cells Using dsRNA Targeted to MLH1 and MSH2

Experiments were performed using the mutagenic nucleobase PB124 (5′A*T*C*A*TCCTACGTTGCACTTG*A*C*C*G [SEQ ID NO 3]). It corresponds to the non-transcribed strand of the SurB gene from tobacco that encodes an ortholog of acetolactate synthase (ALS). The oligonucleotide contains a single mismatch with SurB (underlined) that drives the SurB Proline 191 to glutamic acid conversion, conferring a dominant resistance phenotype to the sulfonylurea type herbicides. The asterisks represent phosphorothioate linkages in which a non-bridging oxygen atom in the phosphate linkage is substituted by a sulphur atom. Such modified linkages are known to be more resistant to exonuclease attack and thus prolong the lifetime of the mutagenic nucleobase in the cell.

Tobacco protoplasts were prepared as described in Example 3. 12.5 μg ds RNA and 10 μg PB124 were transfected to an aliquot of protoplasts and which were finally resuspended in 1.25 ml of T0 culture medium. The suspension was transferred to a 35 mm Petri dish. An equal volume of T0 agarose medium is added and gently mixed. Samples were incubated at 25° C. in the dark and further cultivated as described below.

Tobacco Protoplast Isolation and Transformation

In vitro shoot cultures of Nicotiana tabacum cv Petit Havana line SR1 are maintained on MS20 medium with 0.8% Difco agar in high glass jars at 16/8 h photoperiod of 2000 lux at 25° C. and 60-70% RH. MS20 medium is basic Murashige and Skoog's medium (Murashige, T. and Skoog, F., Physiologia Plantarum, 15: 473-497, 1962) containing 2% (w/v) sucrose, no added hormones and 0.8% Difco agar. Fully expanded leaves of 3-6 week old shoot cultures are harvested. The leaves are sliced into 1 mm thin strips, which are then transferred to large (100 mm×100 mm) Petri dishes containing 45 ml MDE basal medium for a preplasmolysis treatment of 30 min. MDE basal medium contained 0.25 g KCl, 1.0 g MgSO4.7H2O, 0.136 g of KH2PO4, 2.5 g polyvinylpyrrolidone (MW 10,000), 6 mg naphthalene acetic acid and 2 mg 6-benzylaminopurine in a total volume of 900 ml. The osmolality of the solution is adjusted to 600 mOsm.kg-1 with sorbitol, the pH to 5.7. 5 mL of enzyme stock SR1 are then added. The enzyme stock consists of 750 mg Cellulase Onozuka R10, 500 mg driselase and 250 mg macerozyme R10 per 100 ml, filtered over Whatman paper and filter-sterilized. Digestion is allowed to proceed overnight in the dark at 25° C. The digested leaves are filtered through 50 pm nylon sieves into a sterile beaker. An equal volume of cold KCl wash medium is used to wash the sieve and pooled with the protoplast suspension. KCl wash medium consisted of 2.0 g CaCl2.2H2O per liter and a sufficient quantity of mannitol to bring the osmolality to 540 mOsm.kg-1. The suspension is transferred to 10 mL tubes and protoplasts are pelleted for 10 min at 85×g at 4° C. The supernatant is discarded and the protoplast pellets carefully resuspended into 5 mL cold MLm wash medium, which is the macro-nutrients of MS medium (Murashige, T. and Skoog, F., Physiologia Plantarum, 15: 473-497, 1962) at half the normal concentration, 2.2 g of CaCl2.2H2O per liter and a quantity of mannitol to bring the osmolality to 540 mOsm.kg-1. The content of 2 tubes is combined and centrifuged for 10 min at 85×g at 4° C. The supernatant is discarded and the protoplast pellets carefully resuspended into 5 mL cold MLs wash medium which is MLm medium with mannitol replaced by sucrose.

The content of 2 tubes is pooled and 1 mL of KCl wash medium added above the sucrose solution care being taken not to disturb the lower phase. Protoplasts are centrifuged for 10 min at 85×g at 4° C. The interphase between the sucrose and the KCl solutions containing the live protoplasts is carefully collected. An equal volume of KCl wash medium is added and carefully mixed. The protoplast density is measured with a haemocytometer.

Introduction of dsRNA and Protoplast Regeneration

The protoplast suspension is centrifuged at 85×g for 10 minutes at 5° C. The supernatant is discarded and the protoplast pellet resuspended to a final concentration of 106 .mL-1 in KCl wash medium. In a 10 mL tube, 250 μL of protoplast suspension +/−12.5 μg dsRNA and 250 μl of PEG solution (40% PEG4000 (Fluka #81240), 0.1M Ca(NO3)2, 0.4M mannitol) are gently but thoroughly mixed. After 20 min. incubation at room temperature, 5 mL cold 0.275 M Ca(NO3)2 is added dropwise. The protoplast suspension is centrifuged for 10 min at 85×g at 4° C. and the supernatant discarded. The protoplast pellet was then carefully resuspended in 2.5 mL To culture medium supplemented with 50 μg.mL-1 cefotaxime and 50 μg.mL-1 vancomycin. TO culture medium contained (per liter, pH 5.7) 950 mg KNO3, 825 mg NH4NO3, 220 mg CaCl2.2H2O, 185 mg MgSO4.7H2O, 85 mg KH2PO4, 27.85 mg FeSO4.7H2O, 37.25 mg Na2EDTA.2H2O, the micro-nutrients according to Heller's medium (Heller, R., Ann Sci Nat Bot Biol Veg 14: 1-223, 1953), vitamins according to Morel and Wetmore's medium (Morel, G. and R. H. Wetmore, Amer. J. Bot. 38: 138-40, 1951), 2% (w/v) sucrose, 3 mg naphthalene acetic acid, 1 mg 6-benzylaminopurine and a quantity of mannitol to bring the osmolality to 540 mOsm.kg-1 and transferred to a 35 mm Petri dish.

Protoplast Cultivation and Regeneration

After 10 days of cultivation, the agarose slab is cut into 6 equal parts and transferred to a Petri dish containing 22.5 mL MAP1AO medium supplemented with 20 nM chlorsulfuron.

This medium consisted of (per liter, pH 5.7) 950 mg KNO3, 825 mg NH4NO3, 220 mg CaC12.2H2O, 185 mg MgSO4.7H2O, 85 mg KH2PO4, 27.85 mg FeSO4.7H2O, 37.25 mg Na2EDTA.2H2O, the micro-nutrients according to Murashige and Skoog's medium (Murashige, T. and Skoog, F., Physiologia Plantarum, 15: 473-497, 1962) at one tenth of the original concentration, vitamins according to Morel and Wetmore's medium (Morel, G. and R. H. Wetmore, Amer. J. Bot. 38: 138-40, 1951), 6 mg pyruvate, 12 mg each of malic acid, fumaric acid and citric acid, 3% (w/v) sucrose, 6% (w/v) mannitol, 0.03 mg naphthalene acetic acid and 0.1 mg 6-benzylaminopurine. Samples are incubated at 25° C. in low light for 6-8 weeks. Growing calli are then transferred to MAP1 medium and allowed to develop for another 2-3 weeks. MAP1 medium has the same composition as MAP1AO medium, with however 3% (w/v) mannitol instead of 6%, and 46.2 mg.l-1 histidine (pH 5.7). It was solidified with 0.8% (w/v) Difco agar.

Calli are then transferred to RP medium using sterile forceps. RP medium consisted of (per liter, pH 5.7) 273 mg KNO3, 416 mg Ca(NO3)2.4H2O, 392 mg Mg(NO3)2.6H2O, 57 mg MgSO4.7H2O, 233 mg (NH4)2SO4, 271 mg KH2PO4, 27.85 mg FeSO4.7H2O, 37.25 mg Na2EDTA.2H2O, the micro-nutrients according to Murashige and Skoog's medium at one fifth of the published concentration, vitamins according to Morel and Wetmore's medium (Morel, G. and R. H. Wetmore, Amer. J. Bot. 38: 138-40, 1951), 0.05% (w/v) sucrose, 1.8% (w/v) mannitol, 0.25 mg zeatin and 41 nM chlorsulfuron, and is solidified with 0.8% (w/v) Difco agar. Mature shoots are transferred to rooting medium after 2-3 weeks.

### Results

The results of the TGA experiments are shown in Table 6.

TABLE 6
TGA experiments in tobacco protoplasts using dsRNA to down regulate
endogenous MLH1 and MSH2 expression

| | Mutagenic | # chlorsulfuron resistant calli |
|---|---|---|
| dsRNA | nucleobase | per 1 × 10⁶ protoplasts |
| − | − | 0 |
| − | + | 6 |
| + | − | 0 |
| + | + | 192 |

Down regulation of tobacco MLH1 and MSH2 increases the TGA efficiency at least 30 fold. Shoots were regenerated from 20 calli treated with mutagenic nucleobase and dsRNA and genotyped. DNA was isolated from these plants and SurB PCR products including the P191 were amplified and sequenced. All plants showed the expected P191Q mutation and so we conclude that TGA had indeed occurred in these lines.

### TGA Experiments in Tomato Using MLH1 dsRNA

These experiments were performed using the mutagenic nucleobases listed in Table 7. In tomato ALS is a multicopy gene, two full length EST's are present in the Plant Transcript Database (http://planta.tigr.org). In our study we have defined transcript TA37274_{—}4081 as ALS1 and transcript TA37275_{—}4081 as ALS2. ALS1 encodes a protein of 659AA while ALS2 encodes a protein of 657AA. ALS1 and ALS2 show 93% and 96% identity at the DNA and protein levels respectively. The two proteins mainly differ in the signal peptide regions of the proteins responsible for chloroplast targeting. Despite these differences, both ALS1 and ALS2 proteins are both predicted to be targeted to the chloroplast. Previous studies have shown that several amino acid changes at conserved residues of ALS are sufficient to confer a semi-dominant resistance to the sulfonylurea class of herbicides. One of these is the P184Q change. We have previously found that the TGA efficiency in tomato protoplasts is enhanced 8 fold when C5-propyne and LNA (locked nucleic acid) modifications are included on the mutagenic nucleobase. Thus, in this study we have introduced normal DNA mutagenic nucleobases or C5-propyne and LNA modified mutagenic nucleobases designed to produce a P184Q alteration in ALS2 simultaneously with dsRNA targeting tomato MLH1 and MSH2 into tomato leaf protoplasts.

TABLE 7

| SEQ ID | Oligo | Sequence | Mutation |
|---|---|---|---|
| 4 | 95 | A*T*C*A*TCCTCCTCTGCACTTG*A*C*C*G | P184Q |
| 5 | 44 | A*z*y*A*zyyzyyvyTGwAyzzG*A*y*y*G | P184Q |
| 6 | 80 | G*z*A*y*GzAyAxzCAxzAyGzA*G*G*A*U | None |

y = 5-propynyl-2′-deoxycytidine; z = 5-propynyl-2′-deoxyuracil; s = LNA A; v = LNA T; w = LNA C; x = LNA G; U = deoxyuracil; * = phosphorothioate linkages.
All mutagenic nucleobases were synthesized by Eurogentec and HPLC purified.
The nucleotide that forms a mismatch with the target is underlined.

Tomato protoplasts were isolated and transfected as described in example 1. After 7 days the embedded protoplasts were placed on selection medium. Alginate discs were cut up into 5 mm broad strips and layered on TM-DB callus induction medium containing 20 nM chlorsulfuron. Herbicide resistant calli appeared after 4-5 weeks incubation at 30° C., and individuals were then transferred to GM-ZG shooting medium containing 20 nM chlorsulfuron for further growth.

### Results

The results of the TGA experiments using dsRNA to down regulate MLH1 mRNA levels are shown in Table 8.

TABLE 8
dsRNA targeting PMS1 enhances the TGA efficiency
in tomato mesophyll protoplasts

| Mutagenic | | # chlorsulfuron resistant calli |
|---|---|---|
| nucleobase | dsRNA | per 1 × 10⁶ protoplasts |
| 95 | − | 1 |
| 95 | + | 37 |
| 44 | − | 5 |
| 44 | + | 186 |
| 80 | − | 0 |
| 80 | + | 0 |

As in tobacco, transient down regulation of MLH1 by dsRNA in tomato enhances the TGA frequency approximately 30 fold. Analysis of the region of the ALS2 gene containing the P184 codon demonstrated that the herbicide resistant calli did indeed have the expected P184Q alteration.

Overview of (Modified) Nucleotides:

| SEQ | | | |
|---|---|---|---|
| ID NO | Oligo. | Sequence | Modification (position) |
| 1 | | AGCAGTTCCCTTCAGCAAAAAT | |
| 2 | | GAATCGGCGGTATCATCCTTAT | |
| 3 | | A*T*C*A*TCCTACGTTGCACTTG*A*C*C*G | Phosphorothioate |
| | | | (1, 2, 3, 4, 20, 21, 22, 23) |
| 4 | 95 | A*T*C*A*TCCTCCTCTGCACTTG*A*C*C*G | Phosphorothioate |
| | | | (1, 2, 3, 4, 20, 21, 22, 23) |
| 5 | 44 | A*z*y*A*zyyzyyvyTGwAyzzG*A*y*y*G | Phosphorothioate |
| | | | (1, 2, 3, 4, 20, 21, 22, 23); |
| | | | 5-propyny1-2′-deoxyuracil |
| | | | (2, 5, 8, 18, 19); |
| | | | 5-propyny1-2′-deoxycytidine |
| | | | (3, 6, 7, 9, 10, 12, 17, 22, 23) |
| | | | LNA T (11); LNA C (15); |
| 5 | 44 | A*U*C*A*UCCUCCTCTGCACUUG*A*C*C*G | Phosphorothioate |
| | | | (1, 2, 3, 4, 20, 21, 22, 23); |
| | | | 5-propynyl-2′-deoxyuracil |
| | | | (2, 5, 8, 18, 19); |
| | | | 5-propyny1-2′-deoxycytidine |
| | | | (3, 6, 7, 9, 10, 12, 17, 22, 23) |
| | | | LNA T (11); LNA C (15); |
| 6 | 80 | G*z*A*y*GzAyAxzCAxzAyGzA*G*G*A*U | Phosphorothioate |
| | | | (1, 2, 3, 4, 20, 21, 22, 23); |
| | | | 5-propynyl-2′-deoxyuracil |
| | | | (2, 5, 6, 11, 15, 19); |
| | | | 5-propyny1-2′-deoxycytidine |
| | | | (4, 8, 17) |
| | | | LNA G (10, 14) |
| 6 | 80 | G*U*A*C*GUACAGUCAGUACGUA*G*G*A*U | Phosphorothioate |
| | | | (1, 2, 3, 4, 20, 21, 22, 23); |
| | | | 5-propynyl-2′-deoxyuracil |
| | | | (2, 5, 6, 11, 15, 19); |
| | | | 5-propyny1-2′-deoxycytidine |
| | | | (4, 8, 17) |
| | | | LNA G (10, 14) |
| 7 | | CCTGGTCTATTGGATATTGTTAG | |
| 8 | | GCTTGAGCAGTTCTGTATTC | |
| 9 | | GCAATCAAGGAGGAATCAGAGG | |
| 10 | | CCAGCAGCATCAATCAAGCC | |

TABLE 1
EST's from dicotyledonous plant species showing homology with
mismatch repair genes.

| Plant TA | | Plant TA | % | % |
|---|---|---|---|---|
| Accession | Species | Annotation | Identity | Coverage |
| BQ975588 | Helianthus | DNA mismatch repair protein | 63.64 | 34.38 |
| | annuus | [Petunia hybrida (Petunia)] | | |
| CD850432 | Helianthus | DNA mismatch repair protein, | 95.45 | 54.55 |
| | annuus | putative [Oryza sativa (japonica | | |
| | | cultivar-group)] | | |
| TA23223_3694 | Populus | DNA mismatch repair protein | 74.34 | 46.06 |
| | trichocarpa | [Arabidopsis thaliana (Mouse-ear | | |
| | | cress)] | | |
| TA12477_338618 | Aquilegia | DNA mismatch repair protein | 83.16 | 69.26 |
| | formosa x | MSH2 [Arabidopsis thaliana | | |
| | Aquilegia | (Mouse-ear cress)] | | |
| | pubescens | | | |
| TA14299_338618 | Aquilegia | DNA mismatch repair protein | 76.45 | 54.51 |
| | formosa x | [Petunia hybrida (Petunia)] | | |
| | Aquilegia | | | |
| | pubescens | | | |
| TA17063_338618 | Aquilegia | DNA mismatch repair protein | 80.71 | 67.77 |
| | formosa x | [Arabidopsis thaliana (Mouse-ear | | |
| | Aquilegia | cress)] | | |
| | pubescens | | | |
| TA18178_338618 | Aquilegia | Similarity to mismatch repair | 73.65 | 79.15 |
| | formosa x | protein MutS [Arabidopsis thaliana | | |
| | Aquilegia | (Mouse-ear cress)] | | |
| | pubescens | | | |
| TA18731_338618 | Aquilegia | DNA mismatch repair protein | 81.59 | 49.51 |
| | formosa x | [Petunia hybrida (Petunia)] | | |
| | Aquilegia | | | |
| | pubescens | | | |
| TA19622_338618 | Aquilegia | DNA mismatch repair protein-like | 73.16 | 61.16 |
| | formosa x | [Oryza sativa (japonica cultivar- | | |
| | Aquilegia | group)] | | |
| | pubescens | | | |
| DR927199 | Aquilegia | DNA mismatch repair protein | 82.63 | 71.43 |
| | formosa x | MSH6-2 [Arabidopsis thaliana | | |
| | Aquilegia | (Mouse-ear cress)] | | |
| | pubescens | | | |
| DR927200 | Aquilegia | Putative DNA mismatch repair | 64.71 | 83.61 |
| | formosa x | protein [Oryza sativa (japonica | | |
| | Aquilegia | cultivar-group)] | | |
| | pubescens | | | |
| DR934539 | Aquilegia | DNA mismatch repair protein | 83.81 | 39.67 |
| | formosa x | [Lycopersicon esculentum | | |
| | Aquilegia | (Tomato)] | | |
| | pubescens | | | |
| DT730699 | Aquilegia | DNA mismatch repair protein | 73.94 | 99.88 |
| | formosa x | [Petunia hybrida (Petunia)] | | |
| | Aquilegia | | | |
| | pubescens | | | |
| DT739239 | Aquilegia | DNA mismatch repair protein [Zea | 76.19 | 58.13 |
| | formosa x | mays (Maize)] | | |
| | Aquilegia | | | |
| | pubescens | | | |
| TA10253_4236 | Lactuca sativa | DNA mismatch repair protein | 71.08 | 22.27 |
| | | MSH6-2 [Arabidopsis thaliana | | |
| | | (Mouse-ear cress)] | | |
| BQ850805 | Lactuca sativa | DNA mismatch repair protein | 75.24 | 48.61 |
| | | MSH3 [Arabidopsis thaliana | | |
| | | (Mouse-ear cress)] | | |
| CV699936 | Lactuca sativa | DNA mismatch repair protein, | 89.66 | 55.06 |
| | | putative [Oryza sativa (japonica | | |
| | | cultivar-group)] | | |
| DY969585 | Lactuca sativa | DNA mismatch repair protein | 76.87 | 84.28 |
| | | [Petunia hybrida (Petunia)] | | |
| DW342808 | Prunus persica | DNA mismatch repair protein | 77.11 | 32.55 |
| | | MSH6-1 [Arabidopsis thaliana | | |
| | | (Mouse-ear cress)] | | |
| TA14672_35883 | Ipomoea nil | DNA mismatch repair protein | 82.78 | 75.95 |
| | | [Arabidopsis thaliana (Mouse-ear | | |
| | | cress)] | | |
| BQ997162 | Lactuca serriola | DNA mismatch repair protein | 64.65 | 49.42 |
| | | [Arabidopsis thaliana (Mouse-ear | | |
| | | cress)] | | |
| CV255883 | Populus | DNA mismatch repair protein | 70.83 | 75.61 |
| | trichocarpa x | [Petunia hybrida (Petunia)] | | |
| | Populus | | | |
| | deltoides | | | |
| DY816757 | Taraxacum | DNA mismatch repair protein | 74.49 | 96.61 |
| | officinale | [Petunia hybrida (Petunia)] | | |
| BB903729 | Trifolium | DNA mismatch repair protein | 76 | 39.54 |
| | pratense | [Arabidopsis thaliana (Mouse-ear | | |
| | | cress)] | | |
| DW051237 | Lactuca saligna | DNA mismatch repair protein | 81.25 | 33.64 |
| | | MSH2 [Arabidopsis thaliana | | |
| | | (Mouse-ear cress)] | | |
| DW066437 | Lactuca saligna | Similarity to mismatch repair | 74.58 | 85.11 |
| | | protein MutS [Arabidopsis thaliana | | |
| | | (Mouse-ear cress)] | | |
| TA2866_43195 | Lactuca | DNA mismatch repair protein | 76.67 | 93.28 |
| | perennis | [Petunia hybrida (Petunia)] | | |
| DW077683 | Lactuca | DNA mismatch repair protein, | 78.57 | 76.36 |
| | perennis | putative [Oryza sativa (japonica | | |
| | | cultivar-group)] | | |
| DW085719 | Lactuca | DNA mismatch repair protein | 77.03 | 100 |
| | perennis | [Arabidopsis thaliana (Mouse-ear | | |
| | | cress)] | | |
| BF597154 | Glycine soja | DNA mismatch repair protein | 79.41 | 96 |
| | | [Petunia hybrida (Petunia)] | | |
| CV515122 | Mimulus | DNA mismatch repair protein | 78.57 | 30.97 |
| | guttatus | [Arabidopsis thaliana (Mouse-ear | | |
| | | cress)] | | |
| CV517496 | Mimulus | DNA mismatch repair protein | 85.22 | 99.71 |
| | guttatus | MSH2 [Arabidopsis thaliana | | |
| | | (Mouse-ear cress)] | | |
| CV517566 | Mimulus | DNA mismatch repair protein | 76.52 | 61.3 |
| | guttatus | [Petunia hybrida (Petunia)] | | |
| EB693746 | Nicotiana | DNA mismatch repair protein | 88.7 | 81.85 |
| | langsdorffii x | [Petunia hybrida (Petunia)] | | |
| | Nicotiana | | | |
| | sanderae | | | |
| EB713270 | Linum | DNA mismatch repair protein | 65.52 | 82.86 |
| | usitatissimum | MSH6-2 [Arabidopsis thaliana | | |
| | | (Mouse-ear cress)] | | |
| CV461729 | Ribes | DNA mismatch repair protein | 69.72 | 91.73 |
| | americanum | MSH6-2 [Arabidopsis thaliana | | |
| | | (Mouse-ear cress)] | | |
| TA34797_3750 | Malus x | DNA mismatch repair protein | 90.86 | 94.71 |
| | domestica | [Petunia hybrida (Petunia)] | | |
| TA34798_3750 | Malus x | DNA mismatch repair protein | 83.59 | 93.43 |
| | domestica | [Petunia hybrida (Petunia)] | | |
| TA45111_3750 | Malus x | DNA mismatch repair protein | 76.71 | 46.6 |
| | domestica | [Petunia hybrida (Petunia)] | | |
| TA48332_3750 | Malus x | DNA mismatch repair protein | 74.07 | 75.58 |
| | domestica | MSH3 [Arabidopsis thaliana | | |
| | | (Mouse-ear cress)] | | |
| CN874756 | Malus x | DNA mismatch repair protein | 64.29 | 97.67 |
| | domestica | MSH3 [Arabidopsis thaliana | | |
| | | (Mouse-ear cress)] | | |
| CN901049 | Malus x | DNA mismatch repair protein | 73.68 | 95.96 |
| | domestica | [Lycopersicon esculentum | | |
| | | (Tomato)] | | |
| CN926250 | Malus x | DNA mismatch repair protein | 80.95 | 95.45 |
| | domestica | MutS2-like [Arabidopsis thaliana | | |
| | | (Mouse-ear cress)] | | |
| CN930736 | Malus x | DNA mismatch repair protein | 72.17 | 90.79 |
| | domestica | MSH6-1 [Arabidopsis thaliana | | |
| | | (Mouse-ear cress)] | | |
| CN931496 | Malus x | DNA mismatch repair protein | 87.01 | 96.65 |
| | domestica | [Lycopersicon esculentum | | |
| | | (Tomato)] | | |
| CN948744 | Malus x | DNA mismatch repair protein | 84.97 | 98.81 |
| | domestica | [Petunia hybrida (Petunia)] | | |
| CO540662 | Malus x | DNA mismatch repair protein | 81.82 | 90.83 |
| | domestica | MSH6-2 [Arabidopsis thaliana | | |
| | | (Mouse-ear cress)] | | |
| CO753578 | Malus x | DNA mismatch repair protein | 82.76 | 69.88 |
| | domestica | MSH2 [Arabidopsis thaliana | | |
| | | (Mouse-ear cress)] | | |
| EB148408 | Malus x | DNA mismatch repair protein | 83.33 | 55.98 |
| | domestica | MSH2 [Arabidopsis thaliana | | |
| | | (Mouse-ear cress)] | | |
| BE325599 | Medicago | DNA mismatch repair protein | 86.15 | 70.78 |
| | truncatula | MSH2 [Arabidopsis thaliana | | |
| | | (Mouse-ear cress)] | | |
| BF004260 | Medicago | DNA mismatch repair protein | 65.33 | 69.66 |
| | truncatula | [Glycine max (Soybean)] | | |
| BI308552 | Medicago | DNA mismatch repair protein | 64.71 | 60 |
| | truncatula | MSH3 [Arabidopsis thaliana | | |
| | | (Mouse-ear cress)] | | |
| CX539527 | Medicago | DNA mismatch repair protein | 83.25 | 99.66 |
| | truncatula | MSH2 [Arabidopsis thaliana | | |
| | | (Mouse-ear cress)] | | |
| TA39944_4113 | Solanum | DNA mismatch repair protein | 73.33 | 64.58 |
| | tuberosum | MSH6-2 [Arabidopsis thaliana | | |
| | | (Mouse-ear cress)] | | |
| TA43664_4113 | Solanum | DNA mismatch repair protein | 87.76 | 82.87 |
| | tuberosum | [Petunia hybrida (Petunia)] | | |
| TA46125_4113 | Solanum | DNA mismatch repair protein | 74.36 | 46.89 |
| | tuberosum | MSH6-2 [Arabidopsis thaliana | | |
| | | (Mouse-ear cress)] | | |
| TA46208_4113 | Solanum | DNA mismatch repair protein | 83.64 | 21.1 |
| | tuberosum | MutS2-like [Arabidopsis thaliana | | |
| | | (Mouse-ear cress)] | | |
| BE924528 | Solanum | DNA mismatch repair protein | 94.64 | 84 |
| | tuberosum | [Lycopersicon esculentum | | |
| | | (Tomato)] | | |
| BI177912 | Solanum | Similarity to mismatch repair | 70.94 | 48.61 |
| | tuberosum | protein MutS [Arabidopsis thaliana | | |
| | | (Mouse-ear cress)] | | |
| CN213386 | Solanum | DNA mismatch repair protein | 77.04 | 95.92 |
| | tuberosum | [Arabidopsis thaliana (Mouse-ear | | |
| | | cress)] | | |
| DN906164 | Solanum | Putative DNA mismatch repair | 91 | 28.17 |
| | tuberosum | protein [Oryza sativa (japonica | | |
| | | cultivar-group)] | | |
| TA33994_3702 | Arabidopsis | DNA mismatch repair protein | 98.9 | 28.14 |
| | thaliana | MutS2-like [Arabidopsis thaliana | | |
| | | (Mouse-ear cress)] | | |
| TA48304_3702 | Arabidopsis | DNA mismatch repair protein | 100 | 88.95 |
| | thaliana | MSH2 [Arabidopsis thaliana | | |
| | | (Mouse-ear cress)] | | |
| TA48556_3702 | Arabidopsis | DNA mismatch repair protein | 99.88 | 78.26 |
| | thaliana | [Arabidopsis thaliana (Mouse-ear | | |
| | | cress)] | | |
| TA48557_3702 | Arabidopsis | DNA mismatch repair protein | 99.88 | 85.48 |
| | thaliana | [Arabidopsis thaliana (Mouse-ear | | |
| | | cress)] | | |
| TA49938_3702 | Arabidopsis | DNA mismatch repair protein | 100 | 63.18 |
| | thaliana | [Arabidopsis thaliana (Mouse-ear | | |
| | | cress)] | | |
| TA51028_3702 | Arabidopsis | DNA mismatch repair protein | 99.06 | 91.43 |
| | thaliana | MSH3 [Arabidopsis thaliana | | |
| | | (Mouse-ear cress)] | | |
| TA51372_3702 | Arabidopsis | DNA mismatch repair protein | 99.91 | 86.73 |
| | thaliana | [Arabidopsis thaliana (Mouse-ear | | |
| | | cress)] | | |
| TA51817_3702 | Arabidopsis | DNA mismatch repair protein | 99.45 | 94.86 |
| | thaliana | MSH6-2 [Arabidopsis thaliana | | |
| | | (Mouse-ear cress)] | | |
| TA52525_3702 | Arabidopsis | DNA mismatch repair protein | 97.84 | 99.52 |
| | thaliana | MSH6-1 [Arabidopsis thaliana | | |
| | | (Mouse-ear cress)] | | |
| AI994411 | Arabidopsis | Similarity to mismatch repair | 98.92 | 58.12 |
| | thaliana | protein MutS [Arabidopsis thaliana | | |
| | | (Mouse-ear cress)] | | |
| BP643959 | Arabidopsis | DNA mismatch repair protein | 78.43 | 41.58 |
| | thaliana | MSH3 [Arabidopsis thaliana | | |
| | | (Mouse-ear cress)] | | |
| T76569 | Arabidopsis | Similarity to mismatch repair | 95.37 | 78.07 |
| | thaliana | protein MutS [Arabidopsis thaliana | | |
| | | (Mouse-ear cress)] | | |
| W43830 | Arabidopsis | DNA mismatch repair protein | 92.86 | 46.15 |
| | thaliana | [Arabidopsis thaliana (Mouse-ear | | |
| | | cress)] | | |
| TA12097_34305 | Lotus japonicus | DNA mismatch repair protein | 74.03 | 44.34 |
| | | MSH6-2 [Arabidopsis thaliana | | |
| | | (Mouse-ear cress)] | | |
| AV407791 | Lotus japonicus | DNA mismatch repair protein | 75 | 28.8 |
| | | [Petunia hybrids (Petunia)] | | |
| BP043144 | Lotus japonicus | DNA mismatch repair protein-like | 70.77 | 44.42 |
| | | [Oryza sativa (japonica cultivar- | | |
| | | group)] | | |
| BP054691 | Lotus japonicus | DNA mismatch repair protein | 84.57 | 93.46 |
| | | MSH2 [Arabidopsis thaliana | | |
| | | (Mouse-ear cress)] | | |
| EE659074 | Helianthus exilis | DNA mismatch repair protein | 80.72 | 34.2 |
| | | MSH2 [Arabidopsis thaliana | | |
| | | (Mouse-ear cress)] | | |
| EC589850 | Rosa wichurana | DNA mismatch repair protein | 76.53 | 99.22 |
| | | [Petunia hybrida (Petunia)] | | |
| AJ805618 | Antirrhinum | DNA mismatch repair protein | 89.87 | 79.4 |
| | majus | [Petunia hybrida (Petunia)] | | |
| BM172727 | Avicennia | DNA mismatch repair protein | 85 | 81.82 |
| | marina | MSH3 [Arabidopsis thaliana | | |
| | | (Mouse-ear cress)] | | |
| BM062156 | Capsicum | Putative DNA mismatch repair | 69.35 | 30.49 |
| | annuum | protein [Oryza sativa (japonica | | |
| | | cultivar-group)] | | |
| DN625834 | Citrus | DNA mismatch repair protein | 81.54 | 31.45 |
| | aurantium | MutS, C-terminal [Medicago | | |
| | | truncatula (Barrel medic)] | | |
| DY265845 | Citrus | DNA mismatch repair protein | 82.94 | 85.26 |
| | clementina | MSH2 [Zea mays (Maize)] | | |
| DY269901 | Citrus | Similarity to mismatch repair | 76.72 | 94.72 |
| | clementina | protein MutS [Arabidopsis thaliana | | |
| | | (Mouse-ear cress)] | | |
| DY274583 | Citrus | Mismatch repair ATPase MSH4 | 69.55 | 65.43 |
| | clementina | [Aspergillus oryzae] | | |
| DY289119 | Citrus | DNA mismatch repair protein | 75.41 | 31.97 |
| | clementina | MSH6-2 [Arabidopsis thaliana | | |
| | | (Mouse-ear cress)] | | |
| DY298935 | Citrus | DNA mismatch repair protein | 75.94 | 38.07 |
| | clementina | MSH6-2 [Arabidopsis thaliana | | |
| | | (Mouse-ear cress)] | | |
| TA16666_2711 | Citrus sinensis | DNA mismatch repair protein | 83.51 | 45.47 |
| | | [Petunia hybrida (Petunia)] | | |
| TA16667_2711 | Citrus sinensis | DNA mismatch repair protein | 85.58 | 22.32 |
| | | [Petunia hybrida (Petunia)] | | |
| CF509697 | Citrus sinensis | DNA mismatch repair protein | 87.37 | 43.05 |
| | | MutS, C-terminal [Medicago | | |
| | | truncatula (Barrel medic)] | | |
| CK933218 | Citrus sinensis | DNA mismatch repair protein | 79.49 | 43.9 |
| | | MutS, C-terminal [Medicago | | |
| | | truncatula (Barrel medic)] | | |
| CD479592 | Eschscholzia | DNA mismatch repair protein | 73.11 | 81.69 |
| | californica | MSH3 [Arabidopsis thaliana | | |
| | | (Mouse-ear cress)] | | |
| CD669314 | Eucalyptus | DNA mismatch repair protein | 75 | 39.02 |
| | tereticornis | MSH3 [Arabidopsis thaliana | | |
| | | (Mouse-ear cress)] | | |
| DV112264 | Euphorbia esula | DNA mismatch repair protein | 77.94 | 36.76 |
| | | [Petunia hybrida (Petunia)] | | |
| DV134081 | Euphorbia esula | DNA mismatch repair protein | 73.7 | 95.52 |
| | | MSH3 [Arabidopsis thaliana | | |
| | | (Mouse-ear cress)] | | |
| DV134510 | Euphorbia esula | DNA mismatch repair protein | 67.19 | 42.01 |
| | | MutS2-like [Arabidopsis thaliana | | |
| | | (Mouse-ear cress)] | | |
| DV137249 | Euphorbia esula | DNA mismatch repair protein | 78.33 | 23.97 |
| | | [Petunia hybrida (Petunia)] | | |
| DV143230 | Euphorbia esula | Similarity to mismatch repair | 79.25 | 96.92 |
| | | protein MutS [Arabidopsis thaliana | | |
| | | (Mouse-ear cress)] | | |
| BP955997 | Euphorbia | DNA mismatch repair protein | 76.88 | 76.21 |
| | tirucalli | [Petunia hybrida (Petunia)] | | |
| TA61742_3847 | Glycine max | DNA mismatch repair protein | 77.69 | 31.3 |
| | | MutS2-like [Arabidopsis thaliana | | |
| | | (Mouse-ear cress)] | | |
| TA64547_3847 | Glycine max | DNA mismatch repair protein | 99.82 | 86.65 |
| | | [Glycine max (Soybean)] | | |
| TA65430_3847 | Glycine max | DNA mismatch repair protein | 71.03 | 36.9 |
| | | [Petunia hybrida (Petunia)] | | |
| TA69544_3847 | Glycine max | DNA mismatch repair protein | 78.57 | 41.31 |
| | | [Petunia hybrida (Petunia)] | | |
| TA71682_3847 | Glycine max | Putative DNA mismatch repair | 62.5 | 30.16 |
| | | protein [Oryza sativa (japonica | | |
| | | cultivar-group)] | | |
| TA72516_3847 | Glycine max | DNA mismatch repair protein | 74.26 | 53.68 |
| | | [Petunia hybrida (Petunia)] | | |
| TA73086_3847 | Glycine max | DNA mismatch repair protein | 100 | 47.21 |
| | | [Glycine max (Soybean)] | | |
| AW620350 | Glycine max | DNA mismatch repair protein | 92.86 | 98.63 |
| | | [Petunia hybrida (Petunia)] | | |
| AW755937 | Glycine max | DNA mismatch repair protein | 78.92 | 98.4 |
| | | [Petunia hybrida (Petunia)] | | |
| BE020690 | Glycine max | DNA mismatch repair protein | 74.67 | 77.59 |
| | | MSH6-2 [Arabidopsis thaliana | | |
| | | (Mouse-ear cress)] | | |
| BE555291 | Glycine max | DNA mismatch repair protein | 77.68 | 71.95 |
| | | MSH3 [Arabidopsis thaliana | | |
| | | (Mouse-ear cress)] | | |
| BF595206 | Glycine max | DNA mismatch repair protein | 81.4 | 61.58 |
| | | MutS2-like [Arabidopsis thaliana | | |
| | | (Mouse-ear cress)] | | |
| BI321048 | Glycine max | DNA mismatch repair protein | 79.17 | 99.77 |
| | | [Petunia hybrida (Petunia)] | | |
| BM528852 | Glycine max | DNA mismatch repair protein | 82.58 | 78.88 |
| | | MSH2 [Arabidopsis thaliana | | |
| | | (Mouse-ear cress)] | | |
| BU762422 | Glycine max | DNA mismatch repair protein | 79.31 | 75.26 |
| | | MSH6-2 [Arabidopsis thaliana | | |
| | | (Mouse-ear cress)] | | |
| CD391723 | Glycine max | DNA mismatch repair protein | 85.11 | 98.95 |
| | | [Petunia hybrida (Petunia)] | | |
| CD408685 | Glycine max | DNA mismatch repair protein | 76.25 | 85.56 |
| | | [Arabidopsis thaliana (Mouse-ear | | |
| | | cress)] | | |
| BQ406481 | Gossypium | DNA mismatch repair protein | 80.77 | 93.55 |
| | arboreum | [Petunia hybrida (Petunia)] | | |
| CO099019 | Gossypium | DNA mismatch repair protein | 79.17 | 70.76 |
| | raimondii | MutS2-like [Arabidopsis thaliana | | |
| | | (Mouse-ear cress)] | | |
| CO110881 | Gossypium | DNA mismatch repair protein | 77.42 | 30.1 |
| | raimondii | [Petunia hybrida (Petunia)] | | |
| CO110882 | Gossypium | DNA mismatch repair protein | 76.23 | 87.11 |
| | raimondii | [Petunia hybrida (Petunia)] | | |
| CO111184 | Gossypium | DNA mismatch repair protein | 64.77 | 46.23 |
| | raimondii | [Petunia hybrida (Petunia)] | | |
| CO111185 | Gossypium | DNA mismatch repair protein | 75.57 | 98.88 |
| | raimondii | [Petunia hybrida (Petunia)] | | |
| TA4509_73275 | Helianthus | DNA mismatch repair protein | 80.95 | 72.97 |
| | argophyllus | MutS, C-terminal [Medicago | | |
| | | truncatula (Barrel medic)] | | |
| EE614301 | Helianthus | Similarity to mismatch repair | 75.43 | 68.72 |
| | argophyllus | protein MutS [Arabidopsis thaliana | | |
| | | (Mouse-ear cress)] | | |
| CA896912 | Phaseolus | DNA mismatch repair protein | 68.94 | 80.82 |
| | coccineus | MSH6-2 [Arabidopsis thaliana | | |
| | | (Mouse-ear cress)] | | |
| AY795558 | Phaseolus | DNA mismatch repair protein | 100 | 86.61 |
| | vulgaris | [Phaseolus vulgaris (Kidney bean) | | |
| | | (French bean)] | | |
| TA8092_37690 | Poncirus | DNA mismatch repair protein | 83.43 | 67.79 |
| | trifoliata | MSH6-2 [Arabidopsis thaliana | | |
| | | (Mouse-ear cress)] | | |
| CX543480 | Poncirus | Putative DNA mismatch repair | 77.65 | 31.4 |
| | trifoliata | protein [Oryza sativa (japonica | | |
| | | cultivar-group)] | | |
| CX545904 | Poncirus | Similarity to mismatch repair | 81.78 | 86.13 |
| | trifoliata | protein MutS [Arabidopsis thaliana | | |
| | | (Mouse-ear cress)] | | |
| CF228135 | Populus alba x | Similarity to mismatch repair | 79.09 | 58.93 |
| | Populus tremula | protein MutS [Arabidopsis thaliana | | |
| | | (Mouse-ear cress)] | | |
| CF237105 | Populus alba x | DNA mismatch repair protein | 75.38 | 99.67 |
| | Populus tremula | [Petunia hybrida (Petunia)] | | |
| CV130552 | Populus | DNA mismatch repair protein | 72.17 | 37.06 |
| | deltoides | MutS, C-terminal [Medicago | | |
| | | truncatula (Barrel medic)] | | |
| CX169590 | Populus | Similarity to mismatch repair | 77.46 | 77.81 |
| | deltoides | protein MutS [Arabidopsis thaliana | | |
| | | (Mouse-ear cress)] | | |
| TA11495_113636 | Populus tremula | Similarity to mismatch repair | 75.73 | 48.36 |
| | | protein MutS [Arabidopsis thaliana | | |
| | | (Mouse-ear cress)] | | |
| TA23315_47664 | Populus tremula x | DNA mismatch repair protein | 86.11 | 34.07 |
| | Populus | [Petunia hybrida (Petunia)] | | |
| | tremuloides | | | |
| BU809612 | Populus tremula x | Similarity to mismatch repair | 77.14 | 60.58 |
| | Populus | protein MutS [Arabidopsis thaliana | | |
| | tremuloides | (Mouse-ear cress)] | | |
| CV015079 | Rhododendron | DNA mismatch repair protein | 70.37 | 22.85 |
| | catawbiense | MutS2-like [Arabidopsis thaliana | | |
| | | (Mouse-ear cress)] | | |
| BI678389 | Robinia | DNA mismatch repair protein | 90.91 | 42.2 |
| | pseudoacacia | [Petunia hybrida (Petunia)] | | |
| DN168340 | Solanum | DNA mismatch repair protein | 75.35 | 70.53 |
| | habrochaites | MSH6-2 [Arabidopsis thaliana | | |
| | | (Mouse-ear cress)] | | |
| TA4083_64093 | Triphysaria | DNA mismatch repair protein | 65.09 | 93.72 |
| | versicolor | MSH6-2 [Arabidopsis thaliana | | |
| | | (Mouse-ear cress)] | | |
| TA2936_34245 | Zinnia elegans | DNA mismatch repair protein | 71.19 | 51.23 |
| | | MSH6-2 [Arabidopsis thaliana | | |
| | | (Mouse-ear cress)] | | |
| AU302398 | Zinnia elegans | DNA mismatch repair protein [Zea | 69.94 | 93.5 |
| | | mays (Maize)] | | |
| AU308096 | Zinnia elegans | DNA mismatch repair protein | 74.81 | 74.72 |
| | | [Arabidopsis thaliana (Mouse-ear | | |
| | | cress)] | | |
| AU308958 | Zinnia elegans | Similarity to mismatch repair | 80 | 61 |
| | | protein MutS [Arabidopsis thaliana | | |
| | | (Mouse-ear cress)] | | |
| TA50692_29760 | Vitis vinifera | DNA mismatch repair protein | 81.37 | 98.23 |
| | | [Petunia hybrida (Petunia)] | | |
| TA51089_29760 | Vitis vinifera | DNA mismatch repair protein | 82.8 | 25.79 |
| | | [Petunia hybrida (Petunia)] | | |
| EC993306 | Vitis vinifera | DNA mismatch repair protein | 79.05 | 62.98 |
| | | MSH6-2 [Arabidopsis thaliana | | |
| | | (Mouse-ear cress)] | | |
| TA32411_3635 | Gossypium | DNA mismatch repair protein | 73.68 | 48.39 |
| | hirsutum | MSH2 [Arabidopsis thaliana | | |
| | | (Mouse-ear cress)] | | |
| DR454568 | Gossypium | DNA mismatch repair protein | 82.47 | 90.51 |
| | hirsutum | [Petunia hybrida (Petunia)] | | |
| DR460495 | Gossypium | DNA mismatch repair protein | 90.98 | 80.26 |
| | hirsutum | [Petunia hybrida (Petunia)] | | |
| DR461143 | Gossypium | DNA mismatch repair protein | 81.11 | 99.09 |
| | hirsutum | MutS2-like [Arabidopsis thaliana | | |
| | | (Mouse-ear cress)] | | |
| DT455774 | Gossypium | DNA mismatch repair protein | 75.79 | 86.1 |
| | hirsutum | [Lycopersicon esculentum | | |
| | | (Tomato)] | | |
| TA3212_114280 | Cichorium | Cluster: DNA mismatch repair | 64.91 | 22.15 |
| | endivia | protein MutS2-like; n = 1; | | |
| | | Arabidopsis thaliana\|Rep: DNA | | |
| | | mismatch repair protein MutS2-like - | | |
| | | Arabidopsis thaliana (Mouse-ear | | |
| | | cress) | | |
| EH718787 | Centaurea | Cluster: DNA mismatch repair | 79.29 | 79.09 |
| | maculosa | protein; n = 1; Petunia x | | |
| | | hybrida\|Rep: DNA mismatch repair | | |
| | | protein - Petunia hybrida (Petunia) | | |
| EH729317 | Centaurea | Cluster: DNA mismatch repair | 82.11 | 86.17 |
| | maculosa | protein; n = 1; Solanum | | |
| | | lycopersicum\|Rep: DNA mismatch | | |
| | | repair protein - Solanum | | |
| | | lycopersicum (Tomato) | | |
| | | (Lycopersicon esculentum) | | |
| EH775306 | Centaurea | Cluster: DNA mismatch repair | 72.16 | 40.66 |
| | solstitialis | protein; n = 1; Petunia x | | |
| | | hybrida\|Rep: DNA mismatch repair | | |
| | | protein - Petunia hybrida (Petunia) | | |
| AM395588 | Brassica | Cluster: DNA mismatch repair | 84.11 | 96.59 |
| | oleracea | protein; n = 1; Arabidopsis | | |
| | | thaliana\|Rep: DNA mismatch repair | | |
| | | protein - Arabidopsis thaliana | | |
| | | (Mouse-ear cress) | | |
| AM396087 | Brassica | Cluster: DNA mismatch repair | 91.67 | 74.81 |
| | oleracea | protein; n = 1; Arabidopsis | | |
| | | thaliana\|Rep: DNA mismatch repair | | |
| | | protein - Arabidopsis thaliana | | |
| | | (Mouse-ear cress) | | |
| EG983734 | Cyamopsis | Cluster: DNA mismatch repair | 83.76 | 53.51 |
| | tetragonoloba | protein; n = 1; Medicago | | |
| | | truncatula\|Rep: DNA mismatch | | |
| | | repair protein - Medicago | | |
| | | truncatula (Barrel medic) | | |
| EG986084 | Cyamopsis | Cluster: DNA mismatch repair | 75.86 | 27.06 |
| | tetragonoloba | protein MSH6-2; n = 1; Arabidopsis | | |
| | | thaliana\|Rep: DNA mismatch repair | | |
| | | protein MSH6-2 - Arabidopsis | | |
| | | thaliana (Mouse-ear cress) | | |
| CT983366 | Eucalyptus | Cluster: DNA mismatch repair | 75 | 41.31 |
| | gunnii | protein MSH6-2; n = 1; Arabidopsis | | |
| | | thaliana\|Rep: DNA mismatch repair | | |
| | | protein MSH6-2 - Arabidopsis | | |
| | | thaliana (Mouse-ear cress) | | |
| CK645747 | Manihot | Cluster: DNA mismatch repair | 86.13 | 97.74 |
| | esculenta | protein; n = 1; Petunia x | | |
| | | hybrida\|Rep: DNA mismatch repair | | |
| | | protein - Petunia hybrida (Petunia) | | |
| DV447484 | Manihot | Cluster: DNA mismatch repair | 85.19 | 64.46 |
| | esculenta | protein; n = 1; Solanum | | |
| | | lycopersicum\|Rep: DNA mismatch | | |
| | | repair protein - Solanum | | |
| | | lycopersicum (Tomato) | | |
| | | (Lycopersicon esculentum) | | |
| DV451826 | Manihot | Cluster: DNA mismatch repair | 67.61 | 32.22 |
| | esculenta | protein MutS2-like; n = 1; | | |
| | | Arabidopsis thaliana\|Rep: DNA | | |
| | | mismatch repair protein MutS2-like - | | |
| | | Arabidopsis thaliana (Mouse-ear | | |
| | | cress) | | |
| EG562475 | Catharanthus | Cluster: DNA mismatch repair | 70.69 | 31.35 |
| | roseus | protein MutS2-like; n = 1; | | |
| | | Arabidopsis thaliana\|Rep: DNA | | |
| | | mismatch repair protein MutS2-like - | | |
| | | Arabidopsis thaliana (Mouse-ear | | |
| | | cress) | | |
| TA51199_4081 | Solanum | Cluster: DNA mismatch repair | 71.15 | 23.64 |
| | lycopersicum | protein MutS2-like; n = 1; | | |
| | | Arabidopsis thaliana\|Rep: DNA | | |
| | | mismatch repair protein MutS2-like - | | |
| | | Arabidopsis thaliana (Mouse-ear | | |
| | | cress) | | |
| TA54504_4081 | Solanum | Cluster: DNA mismatch repair | 66.67 | 24.93 |
| | lycopersicum | protein MutS2-like; n = 1; | | |
| | | Arabidopsis thaliana\|Rep: DNA | | |
| | | mismatch repair protein MutS2-like - | | |
| | | Arabidopsis thaliana (Mouse-ear | | |
| | | cress) | | |
| AW221187 | Solanum | Cluster: DNA mismatch repair | 99.38 | 99.59 |
| | lycopersicum | protein; n = 1; Solanum | | |
| | | lycopersicum\|Rep: DNA mismatch | | |
| | | repair protein - Solanum | | |
| | | lycopersicum (Tomato) | | |
| | | (Lycopersicon esculentum) | | |
| AW443589 | Solanum | Cluster: DNA mismatch repair | 70.24 | 63.8 |
| | lycopersicum | protein MSH6-2; n = 1; Arabidopsis | | |
| | | thaliana\|Rep: DNA mismatch repair | | |
| | | protein MSH6-2 - Arabidopsis | | |
| | | thaliana (Mouse-ear cress) | | |
| BI931364 | Solanum | Cluster: DNA mismatch repair | 100 | 99.43 |
| | lycopersicum | protein; n = 1; Solanum | | |
| | | lycopersicum\|Rep: DNA mismatch | | |
| | | repair protein - Solanum | | |
| | | lycopersicum (Tomato) | | |
| | | (Lycopersicon esculentum) | | |
| BI932693 | Solanum | Cluster: DNA mismatch repair | 80.43 | 42.01 |
| | lycopersicum | protein MutS2-like; n = 1; | | |
| | | Arabidopsis thaliana\|Rep: DNA | | |
| | | mismatch repair protein MutS2-like - | | |
| | | Arabidopsis thaliana (Mouse-ear | | |
| | | cress) | | |
| BI933338 | Solanum | Cluster: DNA mismatch repair | 82.63 | 80.97 |
| | lycopersicum | protein; n = 1; Medicago | | |
| | | truncatula\|Rep: DNA mismatch | | |
| | | repair protein - Medicago | | |
| | | truncatula (Barrel medic) | | |
| BP879945 | Solanum | Cluster: DNA mismatch repair | 90.57 | 99.17 |
| | lycopersicum | protein; n = 1; Petunia x | | |
| | | hybrida\|Rep: DNA mismatch repair | | |
| | | protein - Petunia hybrida (Petunia) | | |
| BP908253 | Solanum | Cluster: DNA mismatch repair | 100 | 99.45 |
| | lycopersicum | protein; n = 1; Solanum | | |
| | | lycopersicum\|Rep: DNA mismatch | | |
| | | repair protein - Solanum | | |
| | | lycopersicum (Tomato) | | |
| | | (Lycopersicon esculentum) | | |
| DB690287 | Solanum | Cluster: DNA mismatch repair | 99.19 | 72.21 |
| | lycopersicum | protein; n = 1; Solanum | | |
| | | lycopersicum\|Rep: DNA mismatch | | |
| | | repair protein - Solanum | | |
| | | lycopersicum (Tomato) | | |
| | | (Lycopersicon esculentum) | | |
| TA19303_4097 | Nicotiana | Cluster: DNA mismatch repair | 84.78 | 78.26 |
| | tabacum | protein; n = 1; Solanum, | | |
| | | lycopersicum\|Rep: DNA mismatch | | |
| | | repair protein - Solanum | | |
| | | lycopersicum (Tomato) | | |
| | | (Lycopersicon esculentum) | | |
| BP133916 | Nicotiana | Cluster: DNA mismatch repair | 90.48 | 57.53 |
| | tabacum | protein; n = 1; Solanum | | |
| | | lycopersicum\|Rep: DNA mismatch | | |
| | | repair protein - Solanum | | |
| | | lycopersicum (Tomato) | | |
| | | (Lycopersicon esculentum) | | |
| BP526379 | Nicotiana | Cluster: DNA mismatch repair | 72.22 | 90.76 |
| | tabacum | protein; n = 1; Petunia x | | |
| | | hybrida\|Rep: DNA mismatch repair | | |
| | | protein - Petunia hybrida (Petunia) | | |
| BP527860 | Nicotiana | Cluster: DNA mismatch repair | 95.45 | 37.93 |
| | tabacum | protein; n = 1; Petunia x | | |
| | | hybrida\|Rep: DNA mismatch repair | | |
| | | protein - Petunia hybrida (Petunia) | | |
| DV158548 | Nicotiana | Cluster: DNA mismatch repair | 84.88 | 69.57 |
| | tabacum | protein; n = 1; Petunia x | | |
| | | hybrida\|Rep: DNA mismatch repair | | |
| | | protein - Petunia hybrida (Petunia) | | |
| EH367814 | Nicotiana | Cluster: DNA mismatch repair | 69.44 | 43.64 |
| | benthamiana | protein MSH6-2; n = 1; Arabidopsis | | |
| | | thaliana\|Rep: DNA mismatch repair | | |
| | | protein MSH6-2 - Arabidopsis | | |
| | | thaliana (Mouse-ear cress) | | |
| EH371553 | Nicotiana | Cluster: DNA mismatch repair | 75.81 | 70.02 |
| | benthamiana | protein; n = 1; Petunia x | | |
| | | hybrida\|Rep: DNA mismatch repair | | |
| | | protein - Petunia hybrida (Petunia) | | |
| AY650007 | Petunia x | Cluster: DNA mismatch repair | 100 | 91.75 |
| | hybrida | protein; n = 1; Petunia x | | |
| | | hybrida\|Rep: DNA mismatch repair | | |
| | | protein - Petunia hybrida (Petunia) | | |
| AY650008 | Petunia x | Cluster: DNA mismatch repair | 100 | 89.14 |
| | hybrida | protein; n = 1; Petunia x | | |
| | | hybrida\|Rep: DNA mismatch repair | | |
| | | protein - Petunia hybrida (Petunia) | | |
| AY650009 | Petunia x | Cluster: DNA mismatch repair | 100 | 99.06 |
| | hybrida | protein; n = 1; Petunia x | | |
| | | hybrida\|Rep: DNA mismatch repair | | |
| | | protein - Petunia hybrida (Petunia) | | |
| AY650010 | Petunia x | Cluster: DNA mismatch repair | 90.69 | 84.93 |
| | hybrida | protein; n = 1; Petunia x | | |
| | | hybrida\|Rep: DNA mismatch repair | | |
| | | protein - Petunia hybrida (Petunia) | | |
| TA4160_4233 | Helianthus | Cluster: DNA mismatch repair | 70.46 | 76.66 |
| | tuberosus | protein; n = 1; Solanum | | |
| | | lycopersicum\|Rep: DNA mismatch | | |
| | | repair protein - Solanum | | |
| | | lycopersicum (Tomato) | | |
| | | (Lycopersicon esculentum) | | |
| EL443606 | Helianthus | Cluster: DNA mismatch repair | 78.22 | 89.85 |
| | tuberosus | protein; n = 1; Petunia x | | |
| | | hybrida\|Rep: DNA mismatch repair | | |
| | | protein - Petunia hybrida (Petunia) | | |
| TA7350_49390 | Coffea | Cluster: DNA mismatch repair | 77.24 | 27.19 |
| | canephora | protein MSH6-1; n = 1; Arabidopsis | | |
| | | thaliana\|Rep: DNA mismatch repair | | |
| | | protein MSH6-1 - Arabidopsis | | |
| | | thaliana (Mouse-ear cress) | | |
| TA11887_49390 | Coffea | Cluster: DNA mismatch repair | 79.61 | 70.37 |
| | canephora | protein; n = 1; Solanum | | |
| | | lycopersicum\|Rep: DNA mismatch | | |
| | | repair protein - Solanum | | |
| | | lycopersicum (Tomato) | | |
| | | (Lycopersicon esculentum) | | |
| DV680335 | Coffea | Cluster: DNA mismatch repair | 73.98 | 53.4 |
| | canephora | protein MSH6-1; n = 1; Arabidopsis | | |
| | | thaliana\|Rep: DNA mismatch repair | | |
| | | protein MSH6-1 - Arabidopsis | | |
| | | thaliana (Mouse-ear cress) | | |
| DV709725 | Coffea | Cluster: DNA mismatch repair | 86.29 | 59.32 |
| | canephora | protein; n = 1; Medicago | | |
| | | truncatula\|Rep: DNA mismatch | | |
| | | repair protein - Medicago | | |
| | | truncatula (Barrel medic) | | |
| EL418745 | Helianthus | Cluster: DNA mismatch repair | 77.34 | 89.69 |
| | ciliaris | protein MSH6-1; n = 1; Arabidopsis | | |
| | | thaliana\|Rep: DNA mismatch repair | | |
| | | protein MSH6-1 - Arabidopsis | | |
| | | thaliana (Mouse-ear cress) | | |
| EL420651 | Helianthus | Cluster: DNA mismatch repair | 67.42 | 84.78 |
| | ciliaris | protein; n = 1; Petunia x | | |
| | | hybrida\|Rep: DNA mismatch repair | | |
| | | protein - Petunia hybrida (Petunia) | | |
| EX676134 | Fragaria vesca | Cluster: DNA mismatch repair | 76.5 | 89.66 |
| | | protein; n = 1; Medicago | | |
| | | truncatula\|Rep: DNA mismatch | | |
| | | repair protein - Medicago | | |
| | | truncatula (Barrel medic) | | |
| DY674007 | Fragaria vesca | Cluster: Excinuclease ABC, C | 82.53 | 94.24 |
| | | subunit, N-terminal; DNA | | |
| | | mismatch repair protein MutS, C- | | |
| | | terminal; n = 1; Medicago | | |
| | | truncatula\|Rep: Excinuclease ABC, | | |
| | | C subunit, N-terminal; DNA | | |
| | | mismatch repair protein MutS, C- | | |
| | | terminal - Medicago truncatula | | |
| | | (Barrel medic) | | |

1. Method for targeted gene alteration in plant cell protoplasts comprising transfecting the protoplasts with:
a dsRNA that preferably targets plant MMR mRNA; and
a mutagenic nucleobase.

2. Method according to claim 1, wherein the dsRNA and the mutagenic nucleobase are introduced essentially simultaneously into the plant cell protoplasts.

3. Method according to claim 1, wherein the introduction of the dsRNA and the mutagenic nucleobase is at most 48 hours apart.

4. Method according to claim 1, wherein the plant MMR mRNA is the mRNA associated with the MutS and/or MutL MMR genes, more preferably from MSH2, MSH3, MSH6 MSH7 MLH1, MLH2, MLH3 and PMS1.

5. Method according to claim 1, wherein the transfection results in the down regulation of MMR genes, preferably a transient down regulation.

6. Method according to claim 1, wherein the dsRNA is selective in downregulating the MMR system in plant cell protoplasts (does not significantly downregulate other mRNA species in the plant cell protoplasts).

7. Method according to claim 1, wherein the efficiency of the targeted gene alteration increased at least a factor 10 compared to a comparable method for gene alteration in the absence of dsRNA.

8. Method according to claim 1, wherein the plant is selected from amongst monocots and dicots.

9. Method according to claim 7, wherein the plant is a solanacea, preferably tomato and/or tobacco.

10. Method according to claim 1, wherein the mutagenic nucleobase comprises one or more modified nucleotides.

11. Method according to claim 10, wherein the modified nucleotides are selected from the group consisting of:
d. phosphorothioate modifications, preferably near or at one or both ends of the mutagenic nucleobase;
e. propyne substitutions, preferably not near or at one or both ends of the mutagenic nucleobase.
f. LNA substitutions, preferably not near or at one or both ends of the mutagenic nucleobase.

12. Method according to claim 9, wherein the mutagenic nucleobase comprises at least one modified LNA that is positioned at a distance of at least one nucleotide from the at least one mismatch, and wherein, optionally, the mutagenic nucleobase contains at most about 75% LNA modified nucleotides.

13. Method according to claim 9, wherein at least 2, preferably at least 3, more preferably at least 4, even more preferably at least 5 and most preferably at least 6 nucleotides are LNAs.

14. Method according to claim 11, wherein the LNAs are distributed independently over a distance of at most 10 nucleotides, preferably at most 8 nucleotides, more preferably at most 6 nucleotides, even more preferably at most 4, 3, or 2 nucleotides from both sides of the mismatch.

15. Method according to claim 12, wherein 2, preferably 3, more preferably 4, even more preferably 5 and most preferably 6 nucleotides are LNAs.

16. Method according to claim 12, wherein at most 50% of the modified nucleotides of the mutagenic nucleobase are LNA derivatives, preferably at most 40%, more preferably at most 30%, even more preferably at most 20%, and most preferably at most 10%.

17. Method according to claim 10, wherein the at least one modified nucleotide is independently positioned on the 5′ side and/or on the 3′ side of the mismatch.

18. Method according to claim 1, wherein two LNA modified nucleotides located on one side of the 5′ or the 3′ side of the mismatch are separated from each other by at least one, preferably at least two, base pairs.

19. Method according to claim 9, wherein the propyne modified nucleotide is a C7-propyne purine or C5-propyne pyrimidine.

20. Method according to claim 17 , wherein the purine is adenosine or guanosine and/or the pyrimidine is cytosine, uracil or thymidine.

21. Method according to claim 17, wherein at least 10% of the pyrimidines and/or purines are replaced by their respective propynylated derivatives, preferably at least 50%, more preferably at least 75% and most preferably at least 90%.

22. Method according to claim 17, wherein modified nucleotide is a pyrimidine.

23. Method according to claim 17, wherein modified nucleotide is a purine.

24. Method according to claim 1, wherein the nucleotide at the position of the mismatch is not modified.

25. Method according to claim 1, wherein the at least one modified nucleotide is not located adjacent to the mismatch, and preferably is located within 2, 3, 4, 6, 7, 8, 9, or 10 nucleotides of the mismatch.

26. Method according to claim 1, for altering a cell, correcting a mutation by restoration to wild type, inducing a mutation, inactivating an enzyme by disruption of coding region, modifying bioactivity of an enzyme by altering coding region, modifying a protein by disrupting the coding region mismatch repair, targeted alteration of (plant)genetic material, including gene mutation, targeted gene repair and gene knockout.
